SEQUENCE LISTING (15 sequences)

SEQ ID NO: 1 - protein, 21 aa, Artificial Sequence
  Description of Artificial Sequence  Synthetic Peptide
KIKALVEICAELEKEGKISKI

SEQ ID NO: 2 - protein, 101 aa, Artificial Sequence
  Description of Artificial Sequence  Synthetic Peptide
KIKALVEICAELEKEGKISKIKALVEICAALVEICAELVEICAELVEICAELEEICAELEKICAELEKECAELEKEGAELEKEGKELEKEGKILEKEGKIS

SEQ ID NO: 3 - protein, 99 aa, Artificial Sequence
  Description of Artificial Sequence  Synthetic Peptide
IKALVEICAKALVEICAEALVEICAELLVEICAELEVEICAELEKEICAELEKEICAELEKEGCAELEKEGKAELEKEGKIELEKEGKISLEKEGKISK

SEQ ID NO: 4 - protein, 110 aa, Artificial Sequence
  Description of Artificial Sequence  Synthetic Peptide
KIKALVEICAIKALVEICAEKALVEICAELALVEICAELELVEICAELEKVEICAELEKEICAELEKEGKCAELEKEGKIAELEKEGKISELEKEGKISKLEKEGKISKI

SEQ ID NO: 5 - protein, 121 aa, Artificial Sequence
  Description of Artificial Sequence  Synthetic Peptide
KIKALVEICAEIKALVEICAELKALVEICAELEALVEICAELEKLVEICAELEKEVEICAELEKEGEICAELEKEGKICAELEKEGKICAELEKEGKISAELEKEGKISKELEKEGKISKI

SEQ ID NO: 6 - protein, 120 aa, Artificial Sequence
  Description of Artificial Sequence  Synthetic Peptide
KIKALVEICAELIKALVEICAELEKALVEICAELEKALVEICAELEKELVEICAELEKEGVEICAELEKEGKEICAELEKEGKIICAELEKEGKISCAELEKEGKISKAELEKEGKISKI

SEQ ID NO: 7 - protein, 117 aa, Artificial Sequence
  Description of Artificial Sequence  Synthetic Peptide
KIKALVEICAELEIKALVEICAELEKKALVEICAELEKEALVEICAELEKEGLVEICAELEKEGKVEICAELEKEGKIEICAELEKEGKISICAELEKEGKISKCAELEKEGKISKI

SEQ ID NO: 8 - protein, 112 aa, Artificial Sequence
  Description of Artificial Sequence  Synthetic Peptide
KIKALVEICAELEKIKALVEICAELEKEKALVEICAELEKEGALVEICAELEKEGKLVEICAELEKEGKIVEICAELEKEGKISEICAELEKEGKISKICAELEKEGKISKI

SEQ ID NO: 9 - protein, 105 aa, Artificial Sequence
  Description of Artificial Sequence  Synthetic Peptide
KIKALVEICAELEKEIKALVEICAELEKEGKALVEICAELEKEGKALVEICAELEKEGKILVEICAELEKEGKISVEICAELEKEGKISKEICAELEKEGKISKI

SEQ ID NO: 10 - protein, 96 aa, Artificial Sequence
  Description of Artificial Sequence  Synthetic Peptide
KIKALVEICAELEKEGIKALVEICAELEKEGKKALVEICAELEKEGKIALVEICAELEKEGKISLVEICAELEKEGKISKVEICAELEKEGKISKI

SEQ ID NO: 11 - protein, 85 aa, Artificial Sequence
  Description of Artificial Sequence  Synthetic Peptide
KIKALVEICAELEKEGKIKALVEICAELEKEGKIKALVEICAELEKEGKISALVEICAELEKEGKISKLVEICAELEKEGKISKI

SEQ ID NO: 12 - protein, 72 aa, Artificial Sequence
  Description of Artificial Sequence  Synthetic Peptide
KIKALVEICAELEKEGKIIKALVEICAELEKEGKISKALVEICAELEKEGKISKALVEICAELEKEGKISKI

SEQ ID NO: 13 - protein, 57 aa, Artificial Sequence
  Description of Artificial Sequence  Synthetic Peptide
KIKALVEICAELEKEGKISIKALVEICAELEKEGKISKKALVEICAELEKEGKISKI

SEQ ID NO: 14 - protein, 40 aa, Artificial Sequence
  Description of Artificial Sequence  Synthetic Peptide
KIKALVEICAELEKEGKISKIKALVEICAELEKEGKISKI

SEQ ID NO: 15 - protein, 21 aa, Artificial Sequence
  Description of Artificial Sequence  Synthetic Peptide
KIKALVEICAELEKEGKISKI

(19) United States
(12) Patent Application Publication
(10) Pub. No.: US 2005/0074463 A1
(43) Pub. Date: Apr. 7, 2005

(54) Therapeutic vaccination method, mutated peptides of hiv reverse transciptase and their use for vaccination and diagnostic purposes

(75) Inventors: Brigitte Autran, Paris (FR); Assia Samri, Courdimanche (FR); Patrice Debre, Paris (FR); Vincent Calvez, Saint-Mande (FR); Christine Katlama, Paris (FR); Gaby Haas, Bottrop (DE)

Correspondence Address: FULBRIGHT & JAWORSKI L.L.P., 600 CONGRESS AVE., SUITE 2400, AUSTIN, TX, 78701, US

(73) Assignee: Universite Pierre et Marie Curie-paris VI, Paris (FR)

(21) Appl. No.: 10/489,123

(22) PCT Filed: Sep. 14, 2001
(86) PCT No.: PCT/FR01/02872

(51) Int. Cl. A61K 39/00; A61K 39/21

(52) U.S. Cl. 424/185.1; 424/188.1

(57) ABSTRACT

The invention concerns treatment of infectious and tumoral pathologies comprising a anti-infective and/or anti-tumoral chemotherapeutic treatment phase inducing resistance mutations and a therapeutic vaccination phase directed against said resistance mutations and the agents used in said treatment More particularly, the invention concerns peptides of 8 to 80 amino acids of the HIV reverse transcriptase sequence and comprising at least a mutation with respect to said wild sequence of said enzyme, mutation induced in response to treatments by nucleoside and non-nucleoside analogues of the HIV reverse transcriptase. The invention also concerns a pharmaceutical composition or vaccine based on said peptides, for inducing an immune response specific of said mutated sequences and for enhancing or prolonging the efficiency of treatments with nucleoside or non-nucleoside analogues of the HIV reverse transcriptase. The invention further concerns epitopes derived from said peptide sequences to evaluate the specific immune response following the vaccine injection.

The present invention relates to a treatment for infectious and tumor pathologies, comprising a resistance mutation-inducing anti-infectious and/or antitumor chemotherapy treatment phase and a phase of therapeutic vaccination directed against these mutations, and to the agents used in the context of this treatment.

A subject of the present invention is more particularly peptides of 8 to 80 amino acids of the HIV reverse transcriptase sequence and comprising at least one mutation with respect to the wild-type sequence of this enzyme, which mutation is induced in response to treatments with nucleoside or non-nucleoside analogs of the HIV reverse transcriptase.

A subject of the present invention is also a pharmaceutical composition or vaccine based on these peptides, for inducing an immune response specific for these mutated sequences and for enhancing or prolonging the effectiveness of treatments with nucleoside or non-nucleoside analogs of the HIV reverse transcriptase.

A subject of the present invention is also epitopes derived from these peptide sequences for evaluating the specific immune response subsequent to the injection of a vaccine.

The identification of a vaccine which is effective against HIV is essential. An effective immunization would be efficacious and financially reasonable and would constitute a long-term approach to this infection. However, the greatest obstacle to the development of effective vaccines is the variability of HIV. Current data suggest that cytotoxic T lymphocytes (CTLs) are responsible for controlling the initial peak of viremia, the low level viral replication during the asymptomatic phase of the disease and the elimination of most of the viral variants.

The recognition of an infectious agent as foreign supposes that the immune system recognizes certain specific foreign structures, antigens, constituting non-self, by distinguishing them from the structures which belong to it, constituting self. The immune reaction participants involve:
lymphocytes, which are the effector cells of the immune reaction. They comprise B lymphocytes and T lymphocytes subdivided into two subpopulations: CD4+ helper T lymphocytes (Ths), specific immune response coordinators, and CD8+ cytotoxic T lymphocytes (CTLs). These CTLs are capable of recognizing and killing cells infected with viruses, in particular before the virus buds at the surface of the cell and is released into the extracellular medium.
antigen-presenting cells, which capture the antigens, process them and present them in immunogenic form to the T lymphocytes.
MHC (major histocompatibility complex) molecules, which are expressed at the surface of the cells and participate in presenting the antigen to the lymphocytes. In humans, the MHC is called HLA (human leukocyte antigen). Two classes of MHC molecules exist: class I molecules, which are present at the surface of all nuclear cells of the organism, and class II molecules, which are only expressed at the surface of antigen-presenting cells. Antigenic peptides of 8 to 10 amino acids bind in a pocket of the class I MHC molecules, peptides of 14-15 amino acids bind to the class II MHC. Their binding is determined by the forces of interaction which are established between the amino acids (essentially in the two terminal portions) of the antigenic peptide and those of walls of the pocket of the HLA molecules. More than 200 HLA class I alleles have so far been defined within the three families of HLA class I genes, A, B and C. Each HLA class I allele is substantially different within the peptide pocket, and thus the peptides which are able to bind thereto are also different. A strict specificity therefore exists in the interaction between peptide and HLA class I molecule. This specificity explains why even a minor change in the sequence of the peptide presented as target may be sufficient to render this peptide incapable of binding to the HLA class I molecule.
antigen receptors which are attached in the T lymphocyte membrane. By virtue of this receptor, each T lymphocyte specifically recognizes an antigen presented on the MHC. The receptor for T lymphocytes is called TCR (T Cell Receptor). This receptor must recognize, at the same time, at the cell surface, the antigenic peptide and the MHC molecule. The recognition is said to be MHC-restricted.

CD (cluster of differentiation) markers, which are molecules whose presence on the cell membrane identifies a cell. Thus, Ths carry the CD4 marker and are also referred to as CD4+, and the CTLs, which carry the CD8 marker, are referred to as CD8+. CD8+ lymphocytes recognize their antigen on MHC class I; CD4+ lymphocytes on MHC class II.

The presentation of antigenic sequences by the antigen-presenting cells is one of the determining factors of the effectiveness of a vaccine. It can be amplified with certain natural or synthetic compounds, adjuvants, such as lipids (fatty acids, phospholipids, Freund's adjuvant), anionic copolymers, CpG units etc. Other molecules such as cytokines (interleukins, interferons, TNF (tumor necrosis factor), TGF (transforming growth factor, etc.) and chemokines are involved in regulating the maturation, the activation, the proliferation and the differentiation of immune system cells, and play a major role in the moleculation of the vaccine effectiveness.

HIV infection is an infection of the immune system in the sense that the virus electively infects the CD4+ lymphocytes and the antigen-presenting cells and gradually destroys the immune system, generally in 10 years. Some individuals who have been infected for more than 20 years and who have never been treated with antiretroviral treatments have a very low viral load (or a viral load under the limit of detection), however, and exhibit very substantial virus-specific CTL responses directed against HLA class I molecule-restricted epitopes. It has been possible to correlate the intensity and the diversity of these CTL responses to HIV with the control of viral replication, suggesting the essential role played by these CTLs in these immune defenses.

The peptide sequences of HIV which are targets for CTLs have been defined for most HIV proteins. These targets differ according to individuals, as a function of the HLA class I molecules expressed at the surface of the cells of a given individual.

Evasion, by the virus, of the control exerted by the immune system is the main cause of the ineffectiveness of these responses. The viral variation in the CTL epitopes could play an essential role in the immune system's failure to contain the virus. A single mutation in a defined epitope recognized by a CTL may in fact be sufficient for eliminating the CTL recognition, or for blocking the binding of the peptide to the class I molecule, or altering residues essential to the interactions with the TCR. Since the sequence of the HIV reverse transcriptase enzyme is highly conserved between the various viral subtypes, this sequence is a preferred target of CTL-type immune responses: 80% of patients tested exhibit a CTL response directed against the reverse transcriptase corresponding to HIV-1.

A large number of vaccines against HIV have been developed and tested. These vaccines include recombinant HIV proteins, synthetic peptides, recombinant viral or bacterial vectors, and naked DNA-type vaccines. More than 60 phase I clinical trials on about thirty candidates for vaccines specific for HIV type B, which is the most common subtype in the United States and in Europe, have been carried out or are in progress. Most of these trials have focused on the HIV envelope protein. However, the recombinant proteins tested rarely induce CD8+ CTLs which recognize and kill the cells infected with HIV. In addition, the HIV type I isolates exhibit a strong degree of genetic diversity in terms of the viral envelope, which can influence certain aspects of the biological cycle such as infectivity, transmissibility or immunogenicity.

AIDS treatments use molecules capable of blocking one or more steps of the viral cycle without however impairing the correct functioning of the host cells. The other structural proteins (the viral capsid and the reverse transcriptase in particular) and also the regulatory proteins, which are more conserved than the viral envelope, today constitute the preferred targets for preventive vaccination strategies.

Reverse transcriptase (RT), the viral polymerase which catalyzes viral DNA synthesis from the RNA genome, is a target of choice for antiretroviral treatments. Nonhydrolysable analogs of nucleosides (NRTIs), such as in particular AZT (zidovudine), DDI (didanosine), DDC (zalcitabine), 3TC (lamivudine), ABC (abacavir), D4T (stavudine), etc, constitute the family of RT inhibitors most commonly used to date. In triphosphorylated form, they compete with the natural nucleosides during DNA synthesis and bring about termination of the elongation thereof. Another family of inhibitors of the reverse transcription step, the non-nucleoside enzyme inhibitors (NNRTIs), do not require phosphorylation in order to be active and probably act by allosteric inhibition. The most widely known are Nevirapine, Delavirdine or Efavirenz.

The HIV protease constitutes the other main target of antiretroviral treatments which use enzyme inhibitors such as Ritonavir, Saquinavir, Indinavir, etc.

Current antiretroviral therapies generally combine several classes and include one or more reverse transcriptase inhibitors. However, these treatments cannot eradicate the virus and the residual replication which persists in the course of these treatments, even the most active ones, allows the selection, over time, of mutations in the reverse transcriptase gene. Some of these mutations confer on the mutated virus the ability to resist the inhibitory action of these drugs. Thus, many mutations affect in particular the reverse transcriptase sequence (including changes in the active site of the enzyme), thus promoting the emergence of variant viruses resistant to the NRTI or NNRTI families within a period of one to twelve months of treatment. The accumulation of these mutations over time in patients treated with these drugs results in the selection of multiresistant viruses and leads to therapeutic failures.

It has been shown, in patent application WO 99/51750, that treatment with Lamivudine induces point mutations in the catalytic site of HIV reverse transcriptase. This point mutation is located in the site for peptide recognition by the TCR. Consequently, the CTLs of the treated patient which recognized a wild-type epitope were no longer capable of recognizing a mutated epitope.

Latest estimates show that, in western countries, of the 85% of patients infected with HIV, treated with at least one reverse transcriptase inhibitor, 50% of them conserve a detectable viremia and are considered to be in therapeutic failure. This is associated with the existence of at least one mutation of resistance to these inhibitors and results in an increase in the viral load and a decrease in the CD4+ T lymphocytes. In addition, 10% of patients who are recently contaminated, but not yet treated, are contaminated with new viral strains carrying mutations of resistance to antiretroviral treatments. Many studies have shown that subsequent antiviral therapies with molecules of the same class are then less effective on these resistant viruses.

An alternative therapeutic strategy is today proposed in order to limit the toxicity and the cost of these treatments in the long term. It involves stimulating the immune responses to HIV in the treated patients by combining with these treatments anti-HIV immunization with the vaccine candidates available in order, in the end, to interrupt the treatments. Phase I and II clinical trials using vaccine compounds directed against the reference consensus sequences of the nonmutated viruses are in progress.

The applicant has discovered a treatment for infectious and tumor pathologies, comprising a resistance mutation-inducing anti-infectious and/or antitumor chemotherapy treatment phase and a phase of therapeutic vaccination directed against these resistance mutations.

The applicant has discovered more particularly that certain reverse transcriptase sequences, sites of standardized point mutations occurring during various treatments with NRTIs and/or NNRTIs, are in fact capable of causing the induction of specific CTLs capable of recognizing these mutations. The existence of CD8+ cells (of the order of 50 to 500 specific cells per million blood mononuclear cells) directed against several reverse transcriptase mutations induced by NRTIs, such as the mutation 41, 74, 184, etc, in peripheral blood mononuclear cells of treated, infected patients carrying these mutations, has been demonstrated, ex vivo, using methods of the ELISPOT type. This immune response is therefore capable of forming in the course of these treatments, but appears to be incapable of eliminating the specific variants on its own. Amplification of the immunity directed against these mutations will make it possible to control the emergence of these HIV variants and to maintain long-lasting viral suppression with these treatments. The administration of vaccines containing these mutated viral reverse transcriptase sequences, before or during the administration of treatments with NRTIs or NNRTIs should make it possible to prevent, delay or decrease the appearance of mutated viruses capable of withstanding these treatments. This new type of therapeutic immunization is thus aimed at reinforcing or prolonging the effectiveness of antiretroviral treatments with NRTIs and/or NNRTIs. This may be secondarily applied to other mutations induced by the other antiretroviral classes and may also be used in preventive vaccination.

The invention therefore consists of a treatment for infectious and/or tumor pathologies.

The invention also consists of the treatment agents used in the context of this treatment.

A subject of the invention is also the preparation of immunogens containing the mutations for resistance to NRTIs and/or NNRTIS.

More particularly, the invention consists of the preparation of immunogens containing the mutations for resistance to one or more NNRTI(S) used alone or in combination with one or more NRTI(s).

A subject of the invention is also the peptides which are elements of the immunogens.

Another subject of the invention is a pharmaceutical composition, characterized in that it comprises at least one peptide as defined, dispersed in pharmaceutically acceptable excipients, this composition being used as a vaccine.

Another subject of the invention is the determination of epitopes recognized by CTLs specific for the vaccine peptide sequences introduced at vaccination, which can be used for diagnostic purposes.

The method for treating infectious and/or tumor pathologies comprises a resistance mutation-inducing anti-infectious and/or antitumor chemotherapy treatment phase and a phase of therapeutic vaccination directed against these resistance mutations. Preferably, the vaccination phase follows the chemotherapy treatment phase.

The present invention also relates to an agent for treating infectious and/or tumor pathologies, which comprises at least two components:
the first component comprising a resistance mutation—inducing medicinal product intended for the treatment of infectious and/or antitumor pathologies,
the second component comprising a vaccine directed against these resistance mutations, the two components being intended for use simultaneously, separately or sequentially over time.

A subject of the present invention is also peptides of 8 to 80 amino acids of the HIV reverse transcriptase sequence, comprising at least one mutation by simple or multiple substitution and/or insertion with respect to the established sequences of reverse transcriptase, in the absence of any treatment, which mutation is observed subsequent to treatment with NRTIs and/or NNRTIS, the peptides being characterized in that they are capable of inducing a response mediated by T lymphocytes specific for this mutated sequence of HIV reverse transcriptase with the exception of the peptides of sequence Z₁—Y-V-D-D-Z₂, Z₁—Y-1-D-D-Z₂ and Z₁—Y-L-D-D-Z₂, with Z₁ and Z₂ being at least any one amino acid, these peptides having been observed subsequent to treatment with lamivudine alone (NRTI).

In particular, a subject of the invention is preferably peptides of 8 to 80 amino acids of the HIV reverse transcriptase sequence, comprising at least one mutation by simple or multiple substitution and/or insertion with respect to the established sequences of the reverse transcriptase, in the absence of any treatment, which mutation is observed subsequent to treatment with NNRTIs alone or subsequent to treatment combining NNRTIs and NRTIs, the peptides being characterized in that they are capable of inducing a response mediated by T lymphocytes specific for this mutated sequence of HIV reverse transcriptase.

Preferably, the peptides according to the invention comprise from 15 to 50 amino acids.

The sequences of the peptides according to the invention may be preceded or followed by neutral amino acids which can solubilize or stabilize the peptides.

The peptides according to the invention are characterized by the sequences or parts of sequence comprising at least one of the following mutations Mp with Xn being an amino acid corresponding to that described in the Los Alamos databank at the same position in the HIV-1 reverse transcriptase peptide sequence, and n being the position of an amino acid described in the Los Alamos databank in the HIV-1 reverse transcriptase sequence, and with Mp being a mutated amino acid and p being the number of the mutation observed.

* Sequence A:

| | | | | | | | | | | | | | | | |
|---|---|---|---|---|---|---|---|---|---|---|---|---|---|---|---|
| aa | 1 | 2 | 3 | 4 | 5 | 6 | 7 | 8 | 9 | 10 | 11 | 12 | 13 | 14 | 15 |
| | X | X | X | X | X | M1 | X | X | X | X | X | X | X | X | X |

in which M1 (aa6) may be mutated to K, G or S;

| * Sequence B: | | | | | | | | | | | | | | | |
|---|---|---|---|---|---|---|---|---|---|---|---|---|---|---|---|
| aa | 30 | 31 | 32 | 33 | 34 | 35 | 36 | 37 | 38 | 39 | 40 | 41 | 42 | 43 | 44 |
| | X | X | X | X | X | X | X | X | X | M2 | X | M3 | X | X | X |
| | 45 | 46 | 47 | 48 | 49 | 50 | | | | | | | | | |
| | X | X | X | X | X | X | | | | | | | | | |

in which M2 (aa39) may be X39 or may be mutated to A, K, N, P, M or S;
M3 (aa41) may be X41 or may be mutated to L;
   at least one of M2 and M3 representing an amino acid carrying a mutation.
   In a preferred embodiment of the invention, M2 (aa39) may be mutated to A, K, N, P, M or S;

M3 (aa41) may be X41 or may be mutated to L;

| * Sequence C | | | | | | | | | | | | | | | |
|---|---|---|---|---|---|---|---|---|---|---|---|---|---|---|---|
| aa | 1 | 2 | 3 | 4 | 5 | 6 | 7 | 8 | 9 | 10 | 11 | 12 | 13 | 14 | 15 |
| | X | X | X | X | X | M1 | X | X | X | X | X | X | X | X | X |
| | 16 | 17 | 18 | 19 | 20 | 21 | 22 | 23 | 24 | 25 | 26 | 27 | 28 | 29 | 30 |
| | X | X | X | X | X | X | X | X | X | X | X | X | X | X | X |
| | 31 | 32 | 33 | 34 | 35 | 36 | 37 | 38 | 39 | 40 | 41 | 42 | 43 | 44 | 45 |
| | X | X | X | X | X | X | X | X | M2 | X | M3 | X | X | X | X |
| | 46 | 47 | 48 | 49 | 50 | 51 | | | | | | | | | |
| | X | X | X | X | X | X | | | | | | | | | |

in which M1 (aa6) may be X6 or may be mutated to K, G or S;
M2 (aa39) may be X39 or may be mutated to A, K, N, P, M or S;
M3 (aa41) may be X41 or may be mutated to L;
   at least one of M1, M2 and M3 representing an amino acid carrying a mutation.
   In a preferred embodiment of the invention, M1 (aa6) may be X6 or may be mutated to K, G or S;
M2 (aa39) may be X39 or may be mutated to A, K, N, P, M or S;
M3 (aa41) may be X41 or may be mutated to L;

with M1 or M2 representing an amino acid carrying a mutation.

| * Sequence D | | | | | | | | | | | | | | | |
|---|---|---|---|---|---|---|---|---|---|---|---|---|---|---|---|
| aa | 53 | 54 | 55 | 56 | 57 | 58 | 59 | 60 | 61 | 62 | 63 | 64 | 65 | 66 | 67 |
| | X | X | X | X | X | X | X | X | X | M4 | X | X | M5 | X | M6 |
| | 68 | 69 | 70 | 71 | 72 | 73 | 74 | 75 | 76 | 77 | 78 | 79 | 80 | 81 | 82 |
| | M7 | M8 | M9 | X | X | X | M10 | M11 | X | M12 | X | X | X | X | X |
| | 83 | 84 | 85 | 86 | | | | | | | | | | | |
| | X | X | X | X | | | | | | | | | | | |

in which M4 (aa62) may be X62 or may be mutated to V;
M5 (aa65) may be X65 or may be mutated to R;
M6 (aa67) may be X67 or may be mutated to N or G;
M7 (aa68) may be X68 or may be mutated to G or N;
M8 (aa69) may be X69 or may be mutated to D, N, S or A, or combined with multiple insertions such as SG, SX, SSA, SSG, SSS, SEA, STS, ASG, SXX or XXX;
M9 (aa70) may be X70 or may be mutated to R, N, E or S;
M10 (aa74) may be X74 or may be mutated to V or I;
M11 (aa75) may be X75 or may be mutated to M, I, L or T;
M12 (aa77) may be X77 or may be mutated to L; at least one of M4 to M12 representing an amino acid carrying a mutation.
   In a preferred embodiment of the invention, M4 (aa62) may be X62 or may be mutated to V;
M5 (aa65) may be X65 or may be mutated to R;
M6 (aa67) may be X67 or may be mutated to N or G;
M7 (aa68) may be X68 or may be mutated to G or N;
M8 (aa69) may be X69 or may be mutated to D, N, S or A, or combined with multiple insertions such as SG, SX, SSA, SSG, SSS, SEA, STS, ASG, SXX or XXX;
M9 (aa70) may be X70 or may be mutated to R, N, E or S;
M10 (aa74) may be X74 or may be mutated to V or I;
M11 (aa75) may be X75 or may be mutated to M, I, L or T;
M12 (aa77) may be X77 or may be mutated to L;

at least one of M4, M7 or M12 representing an amino acid carrying a mutation.

| * Sequence E | | | | | | | | | | | | | | | |
|---|---|---|---|---|---|---|---|---|---|---|---|---|---|---|---|
| aa | 51 | 52 | 53 | 54 | 55 | 56 | 57 | 58 | 59 | 60 | 61 | 62 | 63 | 64 | 65 |
| | X | X | X | X | X | X | X | X | X | X | X | M4 | X | X | M5 |
| | 66 | 67 | 68 | 69 | 70 | 71 | 72 | 73 | 74 | 75 | 76 | 77 | 78 | 79 | 80 |
| | X | M6 | M7 | M8 | M9 | X | X | X | M10 | M11 | X | M12 | X | X | X |
| | 81 | 82 | 83 | 84 | 85 | 86 | 87 | 88 | 89 | 90 | 91 | 92 | 93 | 94 | 95 |
| | X | X | X | X | X | X | X | M13 | M14 | M15 | X | X | X | X | X |
| | 96 | 97 | 98 | 99 | 100 | 101 | 102 | 103 | 104 | 105 | 106 | 107 | 108 | 109 | 110 |
| | X | X | M16 | X | M17 | M18 | X | M19 | X | X | M20 | X | M21 | X | X |
| | 111 | 112 | 113 | 114 | 115 | 116 | 117 | 118 | 119 | 120 | 121 | 122 | 123 | 124 | 125 |
| | X | X | X | X | M22 | M23 | X | X | M24 | X | X | X | X | | |
| | 126 | 127 | 128 | | | | | | | | | | | | |
| | X | X | X | | | | | | | | | | | | |

in which M4 (aa62) may be X62 or may be mutated to V;
M5 (aa65) may be X65 or may be mutated to R;
M6 (aa67) may be X67 or may be mutated to N or G;
M7 (aa68) may be X68 or may be mutated to G or N;
M8 (aa69) may be X69 or may be mutated to D, N, S or A, or combined with multiple insertions such as SG, SX, SSA, SSG, SSS, SEA, STS, ASG, SXX or XXX;
M9 (aa70) may be X70 or may be mutated to R, N, E or S;
M10 (aa74) may be X74 or may be mutated to V or I;
M11 (aa75) may be X75 or may be mutated to M, I, L or T;
M12 (aa77) may be X77 or may be mutated to L;
M13 (aa88) may be X88 or may be mutated to C;
M14 (aa89) may be X89 or may be mutated to G;
M15 (aa90) may be X90 or may be mutated to I;
M16 (aa98) may be X98 or may be mutated to G or S;
M17 (aa100) may be X100 or may be mutated to I;
M18 (aa101) may be X101 or may be mutated to E, Q, R, I or P;
M19 (aa103) may be X103 or may be mutated to N, Q, R, T or S;
M20 (aa106) may be X106 or may be mutated to A, I or L;
M21 (aa108) may be X108 or may be mutated to I;
M22 (aa115) may be X115 or may be mutated to F;
M23 (aa116) may be X116 or may be mutated to Y;
M24 (aa119) may be X119 or may be mutated to S;
   at least one of M4 to M24 representing an amino acid carrying a mutation.
   In a preferred embodiment of the invention, M4 (aa62) may be X62 or may be mutated to V;
M5 (aa65) may be X65 or may be mutated to R;
M6 (aa67) may be X67 or may be mutated to N or G;
M7 (aa68) may be X68 or may be mutated to G or N;
M8 (aa69) may be X69 or may be mutated to D, N, S or A, or combined with multiple insertions such as SG, SX, SSA, SSG, SSS, SEA, STS, ASG, SXX or XXX;
M9 (aa70) may be X70 or may be mutated to R, N, E or S;
M10 (aa74) may be X74 or may be mutated to V or I;
M11 (aa75) may be X75 or may be mutated to M, I, L or T;
M12 (aa77) may be X77 or may be mutated to L;
M13 (aa88) may be X88 or may be mutated to C;
M14 (aa89) may be X89 or may be mutated to G;
M15 (aa90) may be X90 or may be mutated to I;
M16 (aa98) may be X98 or may be mutated to G or S;
M17 (aa100) may be X100 or may be mutated to I;
M18 (aa101) may be X101 or may be mutated to E, Q, R, I or P;
M19 (aa103) may be X103 or may be mutated to N, Q, R, T or S;
M20 (aa106) may be X106 or may be mutated to A, I or L;
M21 (aa108) may be X108 or may be mutated to I;
M22 (aa115) may be X115 or may be mutated to F;
M23 (aa116) may be X116 or may be mutated to Y;
M24 (aa119) may be X119 or may be mutated to S;

at least one of M4, M7, M12 to M21, M23 or M24 representing an amino acid carrying a mutation.

| * Sequence F | | | | | | | | | | | | | | | |
|---|---|---|---|---|---|---|---|---|---|---|---|---|---|---|---|
| aa | 129 | 130 | 133 | 132 | 133 | 134 | 135 | 136 | 137 | 138 | 139 | 140 | 141 | 142 | 143 |
| | X | X | X | X | X | X | X | X | X | M25 | M26 | X | M27 | X | X |
| | 144 | 145 | 146 | 147 | 148 | 149 | 150 | 151 | 152 | 153 | 154 | 155 | 156 | 157 | 158 |
| | X | X | X | X | X | X | X | M28 | X | X | X | X | X | M29 | X |
| | 159 | 160 | 161 | 162 | 163 | 164 | 165 | 166 | | | | | | | |
| | X | X | X | X | X | X | X | X | | | | | | | |

in which M25 (aa138) may be X138 or may be mutated to A, G, K or Q;
M26 (aa139) may be X139 or may be mutated to I, M or K;
M27 (aa141) may be X141 or may be mutated to E;
M28 (aa151) may be X151 or may be mutated to M;
M29 (aa157) may be X157 or may be mutated to S;
   at least one of M25 to M29 representing an amino acid carrying a mutation.
   In a preferred embodiment of the invention,
M25 (aa138) may be X138 or may be mutated to A, G, K or Q;
M26 (aa139) may be X139 or may be mutated to I, M or K;
M27 (aa141) may be X141 or may be mutated to E;
M28 (aa151) may be X151 or may be mutated to M;
M29 (aa157) may be X157 or may be mutated to S;

at least one of M25 to M28 representing an amino acid carrying a mutation.

| * Sequence G | | | | | | | | | | | | | | | |
|---|---|---|---|---|---|---|---|---|---|---|---|---|---|---|---|
| aa | 163 | 164 | 165 | 166 | 167 | 168 | 169 | 170 | 171 | 172 | 173 | 174 | 175 | 176 | 177 |
| | X | X | X | X | X | X | X | X | X | M30 | X | X | X | X | X |
| | 178 | 179 | 180 | 181 | 182 | 183 | 184 | 185 | 186 | 187 | 188 | 189 | 190 | 191 | 192 |
| | M31 | M32 | X | M33 | X | X | M34 | X | X | X | M35 | M36 | M37 | X | X |
| | 193 | 194 | 195 | 196 | 197 | 198 | 199 | | | | | | | | |
| | X | X | X | X | X | X | X | | | | | | | | |

in which M30 (aa172) may be X172 or may be mutated to K;
M31 (aa178) may be X178 or may be mutated to M, L or F;
M32 (aa179) may be X179 or may be mutated to I, L, D or E;
M33 (aa181) may be X181 or may be mutated to C or I;
M34 (aa184) may be X184 or may be mutated to V, I or T;
M35 (aa188) may be X188 or may be mutated to L, C or H;
M36 (aa189) may be X189 or may be mutated to I;
M37 (aa190) may be X190 or may be mutated to A, S, E, Q or T;
   at least one of M30 to M37 representing an amino acid carrying a mutation.
   In a preferred embodiment of the invention,
M30 (aa172) may be X172 or may be mutated to K;
M31 (aa178) may be X178 or may be mutated to M, L or F;
M32 (aa179) may be X179 or may be mutated to I, L, D or E;
M33 (aa181) may be X181 or may be mutated to C or I;
M34 (aa184) may be X184 or may be mutated to V, I or T;
M35 (aa188) may be X188 or may be mutated to L, C or H;
M36 (aa189) may be X189 or may be mutated to I;
M37 (aa190) may be X190 or may be mutated to A, S, E, Q or T;

at least one of M30, M33, M35 to M37 representing an amino acid carrying a mutation.

| * Sequence H | | | | | | | | | | | | | | | |
|---|---|---|---|---|---|---|---|---|---|---|---|---|---|---|---|
| aa | 169 | 170 | 171 | 172 | 173 | 174 | 175 | 176 | 177 | 178 | 179 | 180 | 181 | 182 | 183 |
| | X | X | X | M30 | X | X | X | X | X | M31 | M32 | X | M33 | X | X |
| | 184 | 185 | | | | | | | | | | | | | |
| | M34 | X | | | | | | | | | | | | | |

in which M30 (aa172) may be X172 or may be mutated to K;
M31 (aa178) may be X178 or may be mutated to M, L or F;
M32 (aa179) may be X179 or may be mutated to I, L, D or E;
M33 (aa181) may be X181 or may be mutated to C or I;
M34 (aa184) may be X184 or may be mutated to V, I or T;
   at least one of M30 to M34 representing an amino acid carrying a mutation.
   In a preferred embodiment of the invention,
M30 (aa172) may be X172 or may be mutated to K;
M31 (aa178) may be X178 or may be mutated to M, L or F;
M32 (aa179) may be X179 or may be mutated to I, L, D or E;
M33 (aa181) may be X181 or may be mutated to C or I;
M34 (aa184) may be X184 or may be mutated to V, I or T;

at least one of M30, M32 or M33 representing an amino acid carrying a mutation.

| * Sequence I | | | | | | | | | | | | | | | | |
|---|---|---|---|---|---|---|---|---|---|---|---|---|---|---|---|---|
| aa | 184 | 185 | 186 | 187 | 188 | 189 | 190 | 191 | 192 | 193 | 194 | 195 | 196 | 197 | 198 | 199 |
| | M34 | X | X | X | M35 | M36 | M37 | X | X | X | X | X | X | X | X | X |

in which M34 (aa184) may be X184;
in which M35 (aa188) may be X188 or may be mutated to L, C or H;
M36 (aa189) may be X189 or may be mutated to I;
M37 (aa190) may be X190 or may be mutated to A, S, E, Q or T;
   at least one of M34 to M37 representing an amino acid carrying a mutation.
   In a preferred embodiment of the invention,
M34 (aa184) may be X184;
   in which M35 (aa188) may be X188 or may be mutated to L, C or H;
M36 (aa189) may be X189 or may be mutated to I;
M37 (aa190) may be X190 or may be mutated to A, S, E, Q or T;

at least one of M35 to M37 representing an amino acid carrying a mutation.

| * Sequence J | | | | | | | | | | | | | | | |
|---|---|---|---|---|---|---|---|---|---|---|---|---|---|---|---|
| aa | 199 | 200 | 201 | 202 | 203 | 204 | 205 | 206 | 207 | 208 | 209 | 210 | 211 | 212 | 213 |
| | X | X | X | X | X | X | X | X | X | M38 | X | M39 | M40 | X | X |
| | 214 | 215 | 216 | 217 | 218 | 219 | 220 | 221 | 222 | 223 | 224 | 225 | 226 | 227 | 228 |
| | M41 | M42 | X | X | X | M43 | X | X | X | M44 | X | M45 | X | M46 | M47 |
| | 229 | 230 | 231 | 232 | 233 | 234 | 235 | 236 | 237 | 238 | 239 | 240 | 241 | 242 | 243 |
| | X | X | X | X | M48 | M49 | X | M50 | X | M51 | X | M52 | X | X | X |
| | 244 | 245 | 246 | 247 | 248 | 249 | | | | | | | | | |
| | X | X | X | X | X | X | | | | | | | | | |

in which M38 (aa208) may be X208 or may be mutated to Y;
M39 (aa210) may be X210 or may be mutated to W;
M40 (aa211) may be X211 or may be mutated to K, A, E, N, D, G or Q;
M41 (aa214) may be X214 or may be mutated to L or F;
M42 (aa215) may be X215 or may be mutated to Y, F or C;
M43 (aa219) may be X219 or may be mutated to Q, E, N, R, T or W;
M44 (aa223) may be X223 or may be mutated to Q;
M45 (aa225) may be X225 or may be mutated to H;
M46 (aa227) may be X227 or may be mutated to L;
M47 (aa228) may be X228 or may be mutated to R, H or F;
M48 (aa233) may be X233 or may be mutated to V or N;
M49 (aa234) may be X234 or may be mutated to I, H or P;
M50 (aa236) may be X236 or may be mutated to M, S or L;
M51 (aa238) may be X238 or may be mutated to T or S;
M52 (aa240) may be X240 or may be mutated to A, D or I;
   at least one of M38 to M52 representing an amino acid carrying a mutation.
   In a preferred embodiment of the invention,
M38 (aa208) may be X208 or may be mutated to Y;
M39 (aa210) may be X210 or may be mutated to W;
M40 (aa211) may be X211 or may be mutated to K, A, E, N, D, G or Q;
M42 (aa215) may be X215 or may be mutated to Y, F or C;
M43 (aa219) may be X219 or may be mutated to Q, E, N, R, T or W;
M44 (aa223) may be X223 or may be mutated to Q;
M45 (aa225) may be X225 or may be mutated to H;
M46 (aa227) may be X227 or may be mutated to L;
M47 (aa228) may be X228 or may be mutated to R, H or F;
M48 (aa233) may be X233 or may be mutated to V or N;
M49 (aa234) may be X234 or may be mutated to I, H or P;
M50 (aa236) may be X236 or may be mutated to M, S or L;
M51 (aa238) may be X238 or may be mutated to T or S;
M52 (aa240) may be X240 or may be mutated to A, D or I;

at least one of M38, M40, M44 to M52 representing an amino acid carrying a mutation.

| * Sequence K | | | | | | | | | | | | | | | |
|---|---|---|---|---|---|---|---|---|---|---|---|---|---|---|---|
| aa | 324 | 325 | 326 | 327 | 328 | 329 | 330 | 331 | 332 | 333 | 334 | 335 | 336 | 337 | 338 |
| | X | X | X | X | X | X | X | X | X | M53 | X | X | X | X | X |
| | 339 | 340 | 341 | 342 | | | | | | | | | | | |
| | X | X | X | X | | | | | | | | | | | |

in which M53 (aa333) may be mutated to E or D.

Preferably, the peptides according to the invention are characterized by the following sequences or parts of sequence comprising at least one of the following mutations Mp:

* Sequence Aa:

| | | | | | | | | | | | | | | | |
|---|---|---|---|---|---|---|---|---|---|---|---|---|---|---|---|
| AA | 1 | 2 | 3 | 4 | 5 | 6 | 7 | 8 | 9 | 10 | 11 | 12 | 13 | 14 | 15 |
| | P | I | S | P | I | M1 | T | V | P | V | K | L | K | P | G |

in which M1 (aa6−wt=E) may be mutated mainly to K, or to G or S;

| * Sequence Ba: | | | | | | | | | | | | | | | |
|---|---|---|---|---|---|---|---|---|---|---|---|---|---|---|---|
| AA | 30 | 31 | 32 | 33 | 34 | 35 | 36 | 37 | 38 | 39 | 40 | 41 | 42 | 43 | 44 |
| | K | I | K | A | L | V | E | I | C | M2 | E | M3 | E | K | E |
| | 45 | 46 | 47 | 48 | 49 | 50 | | | | | | | | | |
| | G | K | I | S | K | I | | | | | | | | | |

in which M2 (aa39−wt=T) may be X39 or may be mutated mainly to A, or to K, or N, or P, or M or S;
M3 (aa41−wt=M) may be X41 or may be mutated mainly to L; at least one of M2 and M3 representing an amino acid carrying a mutation.

In a preferred embodiment of the invention, M2 (aa39) may be mutated to A, K, N, P, M or S; M3 (aa41) may be X41 or may be mutated to L;

| * Sequence Ca: | | | | | | | | | | | | | | | |
|---|---|---|---|---|---|---|---|---|---|---|---|---|---|---|---|
| AA | 1 | 2 | 3 | 4 | 5 | 6 | 7 | 8 | 9 | 10 | 11 | 12 | 13 | 14 | 15 |
| | P | I | S | P | I | M1 | T | V | P | V | K | L | K | P | G |
| | 16 | 17 | 18 | 19 | 20 | 21 | 22 | 23 | 24 | 25 | 26 | 27 | 28 | 29 | 30 |
| | M | D | G | P | K | V | K | Q | W | P | L | T | E | E | K |
| | 31 | 32 | 33 | 34 | 35 | 36 | 37 | 38 | 39 | 40 | 41 | 42 | 43 | 44 | 45 |
| | I | K | A | L | V | E | I | C | M2 | E | M3 | E | K | E | G |
| | 46 | 47 | 48 | 49 | 50 | 51 | | | | | | | | | |
| | K | I | S | K | I | G | | | | | | | | | |

in which M1 (aa6−wt=E) may be mutated mainly to K, or to G or S;
M2 (aa39−wt=T) may be X39 or may be mutated mainly to A, or to K, or N, or P, or M or S;
M3 (aa41−wt=M) may be X41 or may be mutated mainly to L;
   at least one of M1, M2 and M3 representing an amino acid carrying a mutation.
   In a preferred embodiment of the invention, M1 (aa6) may be X6 or may be mutated to K, G or S;
M2 (aa39) may be X39 or may be mutated to A, K, N, P, M or S;

M3 (aa41) may be X41 or may be mutated to L; with M1 or M2 representing an amino acid carrying a mutation.

| * Sequence Da: | | | | | | | | | | | | | | | |
|---|---|---|---|---|---|---|---|---|---|---|---|---|---|---|---|
| AA | 53 | 54 | 55 | 56 | 57 | 58 | 59 | 60 | 61 | 62 | 63 | 64 | 65 | 66 | 67 |
| | E | N | P | Y | N | T | P | V | F | M4 | I | K | M5 | K | M6 |
| | 68 | 69 | 70 | 71 | 72 | 73 | 74 | 75 | 76 | 77 | 78 | 79 | 80 | 81 | 82 |
| | M7 | M8 | M9 | W | R | K | M10 | M11 | D | M12 | R | E | L | N | K |
| | 83 | 84 | 85 | 86 | | | | | | | | | | | |
| | R | T | Q | D | | | | | | | | | | | |

in which M4 (aa62−wt=A) may be X62 or may be mutated mainly to V;
M5 (aa65−wt=K) may be X65 or may be mutated mainly to R;
M6 (aa67−wt=D) may be X67 or may be mutated mainly to N or G;
M7 (aa68−wt=S) may be X68 or may be mutated mainly to G or N;
M8 (aa69−wt=T) may be X69 or may be mutated mainly to D, or to N, or S, or A, or combined with multiple insertions such as SG, SX, SSA, SSG, SSS, SEA, STS, ASG, SXX or XXX;
M9 (aa70−wt=K) may be X70 or may be mutated mainly to R, or to N, or E or S;
M10 (aa74−wt=L) may be X74 or may be mutated mainly to V or to I;
M11 (aa75−wt=V) may be X75 or may be mutated mainly to M, or to I, or L or T;
M12 (aa77−wt=F) may be X77 or may be mutated mainly to L; at least one of M4 to M12 representing an amino acid carrying a mutation.
   In a preferred embodiment of the invention, M4 (aa62) may be X62 or may be mutated to V;
M5 (aa65) may be X65 or may be mutated to R;
M6 (aa67) may be X67 or may be mutated to N or G;
M7 (aa68) may be X68 or may be mutated to G or N;
M8 (aa69) may be X69 or may be mutated to D, N, S or A, or combined with multiple insertions such as SG, SX, SSA, SSG, SSS, SEA, STS, ASG, SXX or XXX;
M9 (aa70) may be X70 or may be mutated to R, N, E or S;
M10 (aa74) may be X74 or may be mutated to V or I;
M11 (aa75) may be X75 or may be mutated to M, I, L or T;
M12 (aa77) may be X77 or may be mutated to L;

at least one of M4, M7 or M12 representing an amino acid carrying a mutation.

| * Sequence Ea: | | | | | | | | | | | | | | | |
|---|---|---|---|---|---|---|---|---|---|---|---|---|---|---|---|
| AA | 51 | 52 | 53 | 54 | 55 | 56 | 57 | 58 | 59 | 60 | 61 | 62 | 63 | 64 | 65 |
| | G | P | E | N | P | Y | N | T | P | V | P | M4 | I | K | M5 |
| | 66 | 67 | 68 | 69 | 70 | 71 | 72 | 73 | 74 | 75 | 76 | 77 | 78 | 79 | 80 |
| | K | M6 | M7 | M8 | M9 | W | R | K | M10 | M11 | D | M12 | R | E | L |
| | 81 | 82 | 83 | 84 | 85 | 86 | 87 | 88 | 89 | 90 | 91 | 92 | 93 | 94 | 95 |
| | N | K | R | T | Q | D | F | M13 | M14 | M15 | Q | L | G | I | P |
| | 96 | 97 | 98 | 99 | 100 | 101 | 102 | 103 | 104 | 105 | 106 | 107 | 108 | 109 | 110 |
| | H | P | M16 | G | M17 | M18 | K | M19 | K | S | M20 | T | M21 | L | D |
| | 111 | 112 | 113 | 114 | 115 | 116 | 117 | 118 | 119 | 120 | 121 | 122 | 123 | 124 | 125 |
| | V | G | D | A | M22 | M23 | S | V | M24 | L | D | E | D | F | R |
| | 126 | 127 | 128 | | | | | | | | | | | | |
| | K | Y | T | | | | | | | | | | | | |

in which M4 (aa62−wt=A) may be X62 or may be mutated mainly to V;
M5 (aa65−wt=K) may be X65 or may be mutated mainly to R;
M6 (aa67−wt=D) may be X67 or may be mutated mainly to N or G;
M7 (aa68−wt=S) may be X68 or may be mutated mainly to G or N;
M8 (aa69−wt=T) may be X69 or may be mutated mainly to D, or to N, or S, or A, or combined with multiple insertions such as SG, SX, SSA, SSG, SSS, SEA, STS, ASG, SXX or XXX;
M9 (aa70−wt=K) may be X70 or may be mutated mainly to R, or to N, or E or S;
M10 (aa74−wt=L) may be X74 or may be mutated mainly to V or to I;
M11 (aa75−wt=V) may be X75 or may be mutated mainly to M, or to I, or L or T;
M12 (aa77−wt=F) may be X77 or may be mutated mainly to L;
M13 (aa88−wt=W) may be X88 or may be mutated mainly to C;
M14 (aa89−wt=E) may be X89 or may be mutated mainly to G;
M15 (aa90−wt=V) may be X90 or may be mutated mainly to I;
M16 (aa98−wt=A) may be X98 or may be mutated mainly to S or to G;
M17 (aa100−wt=L) may be X100 or may be mutated mainly to I;
M18 (aa101−wt=K) may be X101 or may be mutated mainly to E, or to Q, or R, or I or P;
M19 (aa103−wt=K) may be X103 or may be mutated to N, mainly Q or to R, or T or S;
M20 (aa106−wt=V) may be X106 or may be mutated mainly to A, or to I or to L;
M21 (aa108−wt=V) may be X108 or may be mutated mainly to I;
M22 (aa115−wt=Y) may be X115 or may be mutated mainly to F;
M23 (aa116−wt=F) may be X116 or may be mutated mainly to Y;
M24 (aa119−wt=P) may be X119 or may be mutated mainly to S;
   at least one of M4 to M24 representing an amino acid carrying a mutation.
   In a preferred embodiment of the invention, M4 (aa62) may be X62 or may be mutated to V;
M5 (aa65) may be X65 or may be mutated to R;
M6 (aa67) may be X67 or may be mutated to N or G;
M7 (aa68) may be X68 or may be mutated to G or N;
M8 (aa69) may be X69 or may be mutated to D, N, S or A, or combined with multiple insertions such as SG, SX, SSA, SSG, SSS, SEA, STS, ASG, SXX or XXX;
M9 (aa70) may be X70 or may be mutated to R, N, E or S;
M10 (aa74) may be X74 or may be mutated to V or I;
M11 (aa75) may be X75 or may be mutated to M, I, L or T;
M12 (aa77) may be X77 or may be mutated to L;
M13 (aa88) may be X88 or may be mutated to C;
M14 (aa89) may be X89 or may be mutated to G;
M15 (aa90) may be X90 or may be mutated to I;
M16 (aa98) may be X98 or may be mutated to G or S;
M17 (aa100) may be X100 or may be mutated to I;
M18 (aa101) may be X101 or may be mutated to E, Q, R, I or P;
M19 (aa103) may be X103 or may be mutated to N, Q, R, T or S;
M20 (aa106) may be X106 or may be mutated to A, I or L;
M21 (aa108) may be X108 or may be mutated to I;
M22 (aa115) may be X115 or may be mutated to F;
M23 (aa116) may be X116 or may be mutated to Y;
M24 (aa119) may be X119 or may be mutated to S;

at least one of M4, M7, M12 to M21, M23 or M24 representing an amino acid carrying a mutation.

| * Sequence Fa: | | | | | | | | | | | | | | | |
|---|---|---|---|---|---|---|---|---|---|---|---|---|---|---|---|
| AA | 129 | 130 | 131 | 132 | 133 | 134 | 135 | 136 | 137 | 138 | 139 | 140 | 141 | 142 | 143 |
| | A | F | T | I | P | S | I | N | N | M25 | M26 | P | M27 | I | R |
| | 144 | 145 | 146 | 147 | 148 | 149 | 150 | 151 | 152 | 153 | 154 | 155 | 156 | 157 | 158 |
| | Y | Q | Y | N | V | L | P | M28 | G | W | K | G | S | M29 | A |
| | 159 | 160 | 161 | 162 | 163 | 164 | 165 | 166 | | | | | | | |
| | I | F | Q | S | S | M | T | K | | | | | | | |

in which M25 (aa138−wt=E) may be X138 or may be mutated mainly to A, or to G, or K or Q;
M26 (aa139−wt=T) may be X139 or may be mutated mainly to I, or to M or K;
M27 (aa141−wt=G) may be X141 or may be mutated mainly to E;
M28 (aa151−wt=Q) may be X151 or may be mutated mainly to M;
M29 (aa157−wt=P) may be X157 or may be mutated mainly to S;
   at least one of M25 to M29 representing an amino acid carrying a mutation.
   In a preferred embodiment of the invention,
M25 (aa138) may be X138 or may be mutated to A, G, K or Q;
M26 (aa139) may be X139 or may be mutated to I, M or K;
M27 (aa141) may be X141 or may be mutated to E;
M28 (aa151) may be X151 or may be mutated to M;
M29 (aa157) may be X157 or may be mutated to S;

at least one of M25 to M28 representing an amino acid carrying a mutation.

| * Sequence Ga: | | | | | | | | | | | | | | | |
|---|---|---|---|---|---|---|---|---|---|---|---|---|---|---|---|
| AA | 163 | 164 | 165 | 166 | 167 | 168 | 169 | 170 | 171 | 172 | 173 | 174 | 175 | 176 | 177 |
| | S | M | T | K | I | L | E | P | F | M30 | K | Q | N | P | D |
| | 178 | 179 | 180 | 181 | 182 | 183 | 184 | 185 | 186 | 187 | 188 | 189 | 190 | 191 | 192 |
| | M31 | M32 | I | M33 | Q | Y | M34 | D | D | L | M35 | M36 | M37 | S | D |
| | 193 | 194 | 195 | 196 | 197 | 198 | 199 | | | | | | | | |
| | L | E | I | G | Q | H | R | | | | | | | | |

in which M30 (aa172−wt=R) may be X172 or may be mutated mainly to K;
M31 (aa178−wt=I) may be X178 or may be mutated mainly to M, L or to F;
M32 (aa179−wt=V) may be X179 or may be mutated mainly to I, or to L, or D or E;
M33 (aa181−wt=Y) may be X181 or may be mutated mainly to C or to I;
M34 (aa184−wt=M) may be X184 or may be mutated mainly to V, or to I or T;
M35 (aa188−wt=Y) may be X188 or may be mutated mainly to L, or to C or H;
M36 (aa189−wt=V) may be X189 or may be mutated mainly to I;
M37 (aa190−wt=G) may be X190 or may be mutated mainly to A, or to S, or E, or Q or T;
   at least one of M30 to M37 representing an amino acid carrying a mutation.
   In a preferred embodiment of the invention,
M30 (aa172) may be X172 or may be mutated to K;
M31 (aa178) may be X178 or may be mutated to M, L or F;
M32 (aa179) may be X179 or may be mutated to I, L, D or E;
M33 (aa181) may be X181 or may be mutated to C or I;
M34 (aa184) may be X184 or may be mutated to V, I or T;
M35 (aa188) may be X188 or may be mutated to L, C or H;
M36 (aa189) may be X189 or may be mutated to I;
M37 (aa190) may be X190 or may be mutated to A, S, E, Q or T;

at least one of M30, M33, M35 to M37 representing an amino acid carrying a mutation.

| * Sequence Ha: | | | | | | | | | | | | | | | |
|---|---|---|---|---|---|---|---|---|---|---|---|---|---|---|---|
| AA | 169 | 170 | 171 | 172 | 173 | 174 | 175 | 176 | 177 | 178 | 179 | 180 | 181 | 182 | 183 |
| | E | P | F | M30 | K | Q | N | P | D | M31 | M32 | I | M33 | Q | Y |
| | 184 | 185 | | | | | | | | | | | | | |
| | M34 | D | | | | | | | | | | | | | |

in which M30 (aa172−wt=R) may be X172 or may be mutated mainly to K;
M31 (aa178−wt=I) may be X178 or may be mutated mainly to M or to L;
M32 (aa179−wt=V) may be X179 or may be mutated mainly to I, or to L, or D or E;
M33 (aa181−wt=Y) may be X181 or may be mutated mainly to C or to I;
M34 (aa184−wt=M) may be X184 or may be mutated mainly to V, or to I or T;
   at least one of M30 to M34 representing an amino acid carrying a mutation.
   In a preferred embodiment of the invention,
M30 (aa172) may be X172 or may be mutated to K;
M31 (aa178) may be X178 or may be mutated to M, L or F;
M32 (aa179) may be X179 or may be mutated to I, L, D or E;
M33 (aa181) may be X181 or may be mutated to C or I;
M34 (aa184) may be X184 or may be mutated to V, I or T;

at least one of M30; M32 or M r an amino acid carrying a mutation.

| * Sequence Ia: | | | | | | | | | | | | | | | |
|---|---|---|---|---|---|---|---|---|---|---|---|---|---|---|---|
| AA | 184 | 185 | 186 | 187 | 188 | 189 | 190 | 191 | 192 | 193 | 194 | 195 | 196 | 197 | 198 |
| | M34 | D | D | L | M35 | M36 | M37 | S | D | L | E | I | G | Q | H |
| | 199 | | | | | | | | | | | | | | |
| | R | | | | | | | | | | | | | | |

in which M34 (aa184−wt=M) may be X184 or may be mutated mainly to V, or to I or T; in which M35 (aa188−wt=Y) may be X188 or may be mutated mainly to L, or to C or H;
M36 (aa189−wt=V) may be X189 or may be mutated mainly to I;
M37 (aa190−wt=G) may be X190 or may be mutated mainly to A, or to S, or E, or Q or T;
   at least one of M34 to M37 representing an amino acid carrying a mutation.
   In a preferred embodiment of the invention,
M34 (aa184) may be X184;
   in which M35 (aa188) may be X188 or may be mutated to L, C or H;
M36 (aa189) may be X189 or may be mutated to I;
M37 (aa190) may be X190 or may be mutated to A, S, E, Q or T;

at least one of M35 to M37 representing an amino acid carrying a mutation.

| * Sequence Ja: | | | | | | | | | | | | | | | |
|---|---|---|---|---|---|---|---|---|---|---|---|---|---|---|---|
| AA | 199 | 200 | 201 | 202 | 203 | 204 | 205 | 206 | 207 | 208 | 209 | 210 | 211 | 212 | 213 |
| | R | T | K | I | B | E | L | R | Q | M38 | L | M39 | M40 | W | G |
| | 214 | 215 | 216 | 217 | 218 | 219 | 220 | 221 | 222 | 223 | 224 | 225 | 226 | 227 | 228 |
| | M41 | M42 | T | P | D | M43 | K | H | Q | M44 | E | M45 | P | M46 | M47 |
| | 229 | 230 | 231 | 232 | 233 | 234 | 235 | 236 | 237 | 238 | 239 | 240 | 241 | 242 | 243 |
| | W | M | G | Y | M48 | M49 | H | M50 | D | M51 | W | M52 | V | Q | P |
| | 244 | 245 | 246 | 247 | 248 | 249 | | | | | | | | | |
| | I | V | L | P | E | K | | | | | | | | | |

in which M38 (aa208−wt=H) may be X208 or may be mutated mainly to Y;
M39 (aa210−wt=L) may be X210 or may be mutated mainly to W;
M40 (aa211−wt=R) may be X211 or may be mutated mainly to K, or to A, or E, or N, or D, or G, or Q;
M41 (aa214−wt=F or L) may be X214 or may be mutated mainly to L or to F;
M42 (aa215−wt=T) may be X215 or may be mutated mainly to Y, or to F or C;
M43 (aa219−wt=K) may be X219 or may be mutated mainly to Q, or to E, or N, or R, or T or W;
M44 (aa223−wt=K) may be X223 or may be mutated mainly to Q;
M45 (aa225−wt=P) may be X225 or may be mutated mainly to H;
M46 (aa227−wt=F) may be X227 or may be mutated mainly to L;
M47 (aa228−wt=L) may be X228 or may be mutated mainly to R, or to H or F;
M48 (aa233−wt=E) may be X233 or may be mutated mainly to V, or to N;
M49 (aa234−wt=L) may be X234 or may be mutated mainly to I, or to H or P;
M50 (aa236−wt=P) may be X236 or may be mutated mainly to M, or to S or L;
M51 (aa238−wt=K) may be X238 or may be mutated mainly to T or to S;
M52 (aa240−wt=T) may be X240 or may be mutated mainly to A, or to D or I;
   at least one of M38 to M52 representing an amino acid carrying a mutation.
   In a preferred embodiment of the invention,
M38 (aa208) may be X208 or may be mutated to Y;
M39 (aa210) may be X210 or may be mutated to W;
M40 (aa211) may be X211 or may be mutated to K, A, E, N, D, G or Q;
M41 (aa214) may be X214 or may be mutated to L or F;
M42 (aa215) may be X215 or may be mutated to Y, F or C;
M43 (aa219) may be X219 or may be mutated to Q, E, N, R, T or W;
M44 (aa223) may be X223 or may be mutated to Q;
M45 (aa225) may be X225 or may be mutated to H;
M46 (aa227) may be X227 or may be mutated to L;
M47 (aa228) may be X228 or may be mutated to R, H or F;
M48 (aa233) may be X233 or may be mutated to V or N;
M49 (aa234) may be X234 or may be mutated to I, H or P;
M50 (aa236) may be X236 or may be mutated to M, S or L;
M51 (aa238) may be X238 or may be mutated to T or S;
M52 (aa240) may be X240 or may be mutated to A, D or I;

at least one of M38, M40, M44 to M52 representing an amino acid carrying a mutation.

| * Sequence Ka: | | | | | | | | | | | | | | | |
|---|---|---|---|---|---|---|---|---|---|---|---|---|---|---|---|
| AA | 324 | 325 | 326 | 327 | 328 | 329 | 330 | 331 | 332 | 333 | 334 | 335 | 336 | 337 | 338 |
| | D | L | I | A | E | I | Q | K | Q | M53 | Q | G | Q | W | T |
| | 339 | 340 | 341 | 342 | | | | | | | | | | | |
| | Y | Q | I | Y | | | | | | | | | | | |

in which M53 (aa333−wt=G) may be mutated mainly to E or to D.

The invention also relates to a pharmaceutical composition based on at least one peptide, dispersed in pharmaceutically acceptable excipients, for its (their) use as a vaccine.

The pharmaceutical composition according to the invention may also additionally comprise immunomodulators such as cytokines or chemokines.

Adjuvants such as lipids (fatty acids, phospholipids, Freund's incomplete adjuvant in particular), anionic copolymers, CpG units, etc. may be added to the pharmaceutical composition according to the invention.

In one embodiment of the invention, at least one peptide according to the invention makes up the composition of a lipopeptide or of a lipoprotein. In fact, the association of a peptide with a fatty acid or phospholipid sequence makes it possible to observe a potentiation of the immunogenic response compared to that caused by administering the antigen alone.

The peptide(s) according to the invention may be coupled to microparticles or nanoparticles consisting of a polysaccharide core and/or covered in particular with a lipid bilayer. They may also be coupled to one or more liposomes or one or more niosomes.

The peptide(s) according to the invention may be expressed in a recombinant virus, or in a recombinant viral vector, or in an attenuated or inactivated virus.

The peptide(s) according to the invention may be expressed in a recombinant bacterium or in a recombinant bacterial vector.

The peptide(s) according to the invention may be expressed in an antigen-presenting cell.

Another subject of the invention is a vaccine comprising at least one naked DNA or a naked RNA encoding at least one peptide according to the invention.

Another subject of the invention is that the agent for treating patients infected with HIV comprises at least two compositions, the first composition comprising at least one medicinal product of the NRTI and/or NNRTI family, and the second composition comprising at least one peptide according to the invention. These compositions are intended for application sequentially, possibly separately over time, or simultaneously.

Preferably, the agent for treating patients infected with HIV comprises at least two compositions, the first composition comprising at least one medicinal product of the NNRTI family, and the second composition comprising at least one peptide according to the invention.

Another subject of the invention is a method for preventing or treating an HIV virus infection. This method consists in inducing a specific T immune response in response to the administration of a vaccine according to the invention. Preferably, the mutation(s) in the HIV virus are located in the reverse transcriptase enzyme.

Another subject of the invention is peptide sequences of 8 to 20 amino acids, recognized as epitope sequences by T cells specific for the peptide sequences corresponding beforehand to the vaccination, introduced at vaccination.

The epitope sequences according to the invention are peptide sequences of 8 to 20 contiguous amino acids chosen from the peptide sequences A to K having at least one mutation Mp. Preferably, these peptide sequences consist of 8 to 10 contiguous amino acids chosen from the peptide sequences A to K having at least one mutation Mp. More preferably, the epitope sequences according to the invention consist of 9 contiguous amino acids chosen from the peptide sequences A to K having at least one mutation Mp.

The epitope sequences having the stated characteristics can be used for determining and quantifying the T cells specific for these epitope sequences.

A subject of the invention is also a diagnostic composition for determining the T cells specific for these epitope sequences, characterized in that it comprises, inter alia, at least one epitope sequence according to the invention.

The vaccines obtained according to the methods of the invention can be formulated as pharmaceutical compositions for subcutaneous, intramuscular, intradermal or intravenous administration. The techniques for administering this type of vaccine are thoroughly described in the literature and are well known to those skilled in the art.

Pharmaceutically acceptable excipients can be added according to the administration technique used. Aqueous excipients, such as a saline buffer solution, may in particular be added. These solutions are sterile, and free of toxic or pyrogenic substances.

The compositions according to the invention may be sterilized by conventional known sterilization methods.

Depending on the method of administration, the compositions according to the invention may be in all the forms conventionally used in diagnostics.

Compositions according to the invention may in particular contain pharmaceutically acceptable substances such as pH stabilizers, buffer agents or, for example, sodium acetate, sodium chloride, potassium chloride, calcium chloride, sodium lactate or the like, so as to be under physiological conditions.

The following examples illustrate the invention without limiting it in any way.

### EXAMPLE 1

### Generation of CTL Lines

The consequences of mutations induced under NRTIs, on RT-specific CTL responses, were studied. A total of 66 blood samples from 35 patients treated or not treated with mono- or bitherapy with NRTIs, without protease inhibitor, were collected for 1 to 6 years every year between 1991 and 1996.

In order to analyze the recognition of the HIV-1 virus pol and rt gene products, HIV-specific lines were generated by coculturing PBMCs (peripheral blood mononuclear cells) from patients and autologous PHA-blasts. PBMCs stimulated with PHA (phytohemagglutinin) at 2 μg/ml for 18 h at 37° C. in a humid atmosphere at 5% CO₂ and irradiated at 5000 rads are added to freshly thawed autologous PBMCs at a ratio of 1:5 and deposited in 24-well plates at a concentration of 1×10⁶/ml. The CTL lines are cultured for at least 3 weeks in the presence of interleukin 2 at 20 U/ml, added on the third day of culture and then every 3 days. These lines are tested for their cytotoxic activity after at least 3 weeks of culturing, against autologous B-EBV lines (B lymphocytes transformed with EBV) acting as targets.

The same method was used with regard to the mutations induced under NNRTI treatment or under combined NNRTI and NRTI treatment.

### EXAMPLE 2

### CTL Assay

The CTL activity was measured using a ⁵¹chromium release assay. The target cells are autologous B-EBV lines infected with the various recombinant vaccinia viruses expressing the pol gene or fragments of the pol gene of HIV-1, at a multiplicity of infection of 5 PFU (plaque forming unit)/cell, 18 h before the CTL assay. The non-recombined wild-type vaccinia virus (Vac-WT) serves as a control. The target cells are incubated in a pellet in the presence of Na₂⁵¹CrO₄ (specific activity 2 mci/ml), at a concentration of 70 μci/10⁶ cells, for 2 h at 37° C. The radioactivity measured is calculated according to the following formula: (experimental release−spontaneous release)/(total release−spontaneous release)×100=% specific lysis. The CTL responses are considered to be positive when more than 10% specific lysis is obtained, at at least 2 successive effector/target ratios.

The analysis of recognition of the RT-1:1-143 and RT-2:143-293 regions was carried out on Pol-specific CTL lines. The cytotoxic T responses were evaluated using a chromium 51 release assay. The target cells were autologous B-EBV lines infected with vaccinia viruses expressing various rt gene fragments (RT-1:1-143 and RT-2:143-293).

### EXAMPLE 3

### Detection of the Resistance Mutations

The resistance mutations were detected in the proviral DNA using a LiPA HIV-1 RT assay (Murex Diagnostics SA, Châtillon, France). This assay allows simultaneous detection of the “wild-type” sequences and of the resistance mutations (41, 69, 70, 74, 184, 214 and 215) induced by the nucleoside RT inhibitors (NRTIs). Using the DNA isolated from the PBMCs, an amplification of the rt gene is carried out using biotinylated primers, and the biotinylated DNA is then hybridized with specific oligonucleotide probes immobilized in parallel lines on a strip. The addition of alkaline phosphatase-labeled streptavidin results in binding thereof with the biotinylated hydrids formed, which leads to the formation of a violet/brown precipitate. The wild-type and mutated rt sequences located at the various codons can be located on the same strip.

### EXAMPLE 4

### ELISpot Assay

For the recognition of epitope peptides, an ELISpot-γIFN assay in which the PBMCs were incubated, without prior stimulation, with the peptide of interest, was used.

The 96 wells of an Immobilon p plate (Millipore, Molsheim, France) are coated for 18 h at 4° C. with a monoclonal anti-γIFN (γ-interferon) capture antibody (IgG1 B-B1 at 1 μg/ml, Diaclone, Besançon, France). After 3 washes, the PBMCs are added in triplicate at a concentration of 1×10⁵ and 5×10⁴ cells per well in the presence of 5 μg/ml of peptide or 0.5 μg/ml of PHA, used as positive control, or medium alone used as negative control. The plates are then incubated for 40 h at 37° C. in a humid atmosphere at 5% CO₂, and then washed, the biotinylated monoclonal anti-γIFN detection antibody (B-G1 at 0.2 μg/ml, Diaclone, Besançon, France) is then added and the plates are again incubated for 4 h at 37° C. After the washes, alkaline phosphatase-labeled streptavidin (Amersham, Les Ulis, France) is added and the plate is incubated for 1 h at 37° C. The plate is then washed and incubated with BCIP/NTP (Sigma, Aldrich, Saint Quentin, France), and violet spots appear after 10 to 15 min. The plate is washed with water to stop the reaction. The spots are counted using an automatic ELISpot reader. The number of specific responder T cells is calculated after subtraction of the negative control values. The frequency of cells secreting γIFN is given as SFC (spot forming cells).

### EXAMPLE NO. 5

### Fragmentation of the Sequence B (aa 30-50) into Various Peptides Ranging Between 8 and 21 Amino Acids in Length

The peptide sequences are fragmented into peptides of 8 to 80 amino acids (aa) involving any part of the sequence covering at least one mutation, for therapeutic or diagnostic applications.

For the peptide sequence 30-50 where M2 corresponds to mutated aa 39 (A) and M3 corresponds to mutated aa 41 (L):

| aa | 30 | 31 | 32 | 33 | 34 | 35 | 36 | 37 | 38 | 39 | 40 | 41 | 42 | 43 | 44 | 45 | 46 | 47 | 48 | 49 | 50 | |
|---|---|---|---|---|---|---|---|---|---|---|---|---|---|---|---|---|---|---|---|---|---|---|
| | K | I | K | A | L | V | E | I | C | A | E | L | E | K | E | G | K | I | S | K | I | |
| * Sequences of 8aa: | | | | | | | | | | | | | | | | | | | | | | |
| aa 32-39 | 32 | 33 | 34 | 35 | 36 | 37 | 38 | 39 | | | | | | | | | | | | | | |
| | K | A | L | V | E | I | C | A | | | | | | | | | | | | | | |
| aa 33-40 | 33 | 34 | 35 | 36 | 37 | 38 | 39 | 40 | | | | | | | | | | | | | | |
| | A | L | V | E | I | C | A | E | | | | | | | | | | | | | | |
| aa 34-41 | 34 | 35 | 36 | 37 | 38 | 39 | 40 | 41 | | | | | | | | | | | | | | |
| | L | V | E | I | C | A | E | L | | | | | | | | | | | | | | |
| aa 35-42 | 35 | 36 | 37 | 38 | 39 | 40 | 41 | 42 | | | | | | | | | | | | | | |
| | V | E | I | C | A | E | L | E | | | | | | | | | | | | | | |
| aa 36-43 | 36 | 37 | 38 | 39 | 40 | 41 | 42 | 43 | | | | | | | | | | | | | | |
| | E | I | C | A | E | L | E | K | | | | | | | | | | | | | | |
| aa 37-44 | 37 | 38 | 39 | 40 | 41 | 42 | 43 | 44 | | | | | | | | | | | | | | |
| | I | C | A | E | L | E | K | E | | | | | | | | | | | | | | |
| aa 38-45 | 38 | 39 | 40 | 41 | 42 | 43 | 44 | 45 | | | | | | | | | | | | | | |
| | C | A | E | L | E | K | E | G | | | | | | | | | | | | | | |
| aa 39-46 | 39 | 40 | 41 | 42 | 43 | 44 | 45 | 46 | | | | | | | | | | | | | | |
| | A | E | L | E | K | E | G | K | | | | | | | | | | | | | | |
| aa 40-47 | 40 | 41 | 42 | 43 | 44 | 45 | 46 | 47 | | | | | | | | | | | | | | |
| | E | L | E | K | E | G | K | I | | | | | | | | | | | | | | |
| aa 41-48 | 41 | 42 | 43 | 44 | 45 | 46 | 47 | 48 | | | | | | | | | | | | | | |
| | L | E | K | E | G | K | I | S | | | | | | | | | | | | | | |
| * Sequences of 9aa: | | | | | | | | | | | | | | | | | | | | | | |
| aa 31-39 | 31 | 32 | 33 | 34 | 35 | 36 | 37 | 38 | 39 | | | | | | | | | | | | | |
| | I | K | A | L | V | E | I | C | A | | | | | | | | | | | | | |
| aa 32-40 | 32 | 33 | 34 | 35 | 36 | 37 | 38 | 39 | 40 | | | | | | | | | | | | | |
| | K | A | L | V | E | I | C | A | E | | | | | | | | | | | | | |
| aa 33-41 | 33 | 34 | 35 | 36 | 37 | 38 | 39 | 40 | 41 | | | | | | | | | | | | | |
| | A | L | V | E | I | C | A | E | L | | | | | | | | | | | | | |
| aa 34-42 | 34 | 35 | 36 | 37 | 38 | 39 | 40 | 41 | 42 | | | | | | | | | | | | | |
| | L | V | E | I | C | A | E | L | E | | | | | | | | | | | | | |
| aa 35-43 | 35 | 36 | 37 | 38 | 39 | 40 | 41 | 42 | 43 | | | | | | | | | | | | | |
| | V | E | I | C | A | E | L | E | K | | | | | | | | | | | | | |
| aa 36-44 | 36 | 37 | 38 | 39 | 40 | 41 | 42 | 43 | 44 | | | | | | | | | | | | | |
| | E | I | C | A | E | L | E | K | E | | | | | | | | | | | | | |
| aa 37-45 | 37 | 38 | 39 | 40 | 41 | 42 | 43 | 44 | 45 | | | | | | | | | | | | | |
| | I | C | A | E | L | E | K | E | G | | | | | | | | | | | | | |
| aa 38-46 | 38 | 39 | 40 | 41 | 42 | 43 | 44 | 45 | 46 | | | | | | | | | | | | | |
| | C | A | E | L | E | K | E | G | K | | | | | | | | | | | | | |
| aa 39-47 | 39 | 40 | 41 | 42 | 43 | 44 | 45 | 46 | 47 | | | | | | | | | | | | | |
| | A | E | L | E | K | E | G | K | I | | | | | | | | | | | | | |
| aa 40-48 | 40 | 41 | 42 | 43 | 44 | 45 | 46 | 47 | 48 | | | | | | | | | | | | | |
| | E | L | E | K | E | G | K | I | S | | | | | | | | | | | | | |
| aa 41-49 | 41 | 42 | 43 | 44 | 45 | 46 | 47 | 48 | 49 | | | | | | | | | | | | | |
| | L | E | K | E | G | K | I | S | K | | | | | | | | | | | | | |
| * Sequences of 10aa: | | | | | | | | | | | | | | | | | | | | | | |
| aa 30-39 | 30 | 31 | 32 | 33 | 34 | 35 | 36 | 37 | 38 | 39 | | | | | | | | | | | | |
| | K | I | K | A | L | V | E | I | C | A | | | | | | | | | | | | |
| aa 31-40 | 31 | 32 | 33 | 34 | 35 | 36 | 37 | 38 | 39 | 40 | | | | | | | | | | | | |
| | I | K | A | L | V | E | I | C | A | E | | | | | | | | | | | | |
| aa 32-41 | 32 | 33 | 34 | 35 | 36 | 37 | 38 | 39 | 40 | 41 | | | | | | | | | | | | |
| | K | A | L | V | E | I | C | A | E | L | | | | | | | | | | | | |
| aa 33-42 | 33 | 34 | 35 | 36 | 37 | 38 | 39 | 40 | 41 | 42 | | | | | | | | | | | | |
| | A | L | V | E | I | C | A | E | L | E | | | | | | | | | | | | |
| aa 34-43 | 34 | 35 | 36 | 37 | 38 | 39 | 40 | 41 | 42 | 43 | | | | | | | | | | | | |
| | L | V | E | I | C | A | E | L | E | K | | | | | | | | | | | | |
| aa 35-44 | 35 | 36 | 37 | 38 | 39 | 40 | 41 | 42 | 43 | 44 | | | | | | | | | | | | |
| | V | E | I | C | A | E | L | E | K | E | | | | | | | | | | | | |
| aa 36-45 | 36 | 37 | 38 | 39 | 40 | 41 | 42 | 43 | 44 | 45 | | | | | | | | | | | | |
| | E | I | C | A | E | L | E | K | E | G | | | | | | | | | | | | |
| aa 37-46 | 37 | 38 | 39 | 40 | 41 | 42 | 43 | 44 | 45 | 46 | | | | | | | | | | | | |
| | I | C | A | E | L | E | K | E | G | I | | | | | | | | | | | | K |
| aa 38-47 | 38 | 39 | 40 | 41 | 42 | 43 | 44 | 45 | 46 | 47 | | | | | | | | | | | | |
| | C | A | E | L | E | K | E | G | K | I | | | | | | | | | | | | |
| AA 39-48 | 39 | 40 | 41 | 42 | 43 | 44 | 45 | 46 | 47 | 48 | | | | | | | | | | | | |
| | A | E | L | E | K | E | G | K | I | S | | | | | | | | | | | | |
| aa 40-49 | 40 | 41 | 42 | 43 | 44 | 45 | 46 | 47 | 48 | 49 | | | | | | | | | | | | |
| | E | L | E | K | E | G | K | I | S | K | | | | | | | | | | | | |
| aa 41-50 | 41 | 42 | 43 | 44 | 45 | 46 | 47 | 48 | 49 | 50 | | | | | | | | | | | | |
| | L | E | K | E | G | K | I | S | K | I | | | | | | | | | | | | |
| * Sequences of 11aa: | | | | | | | | | | | | | | | | | | | | | | |
| aa 30-40 | 30 | 31 | 32 | 33 | 34 | 35 | 36 | 37 | 38 | 39 | 40 | | | | | | | | | | | |
| | K | I | K | A | L | V | E | I | C | A | E | | | | | | | | | | | |
| aa 31-41 | 31 | 32 | 33 | 34 | 35 | 36 | 37 | 38 | 39 | 40 | 41 | | | | | | | | | | | |
| | I | K | A | L | V | E | I | C | A | E | L | | | | | | | | | | | |
| aa 32-42 | 32 | 33 | 34 | 35 | 36 | 37 | 38 | 39 | 40 | 41 | 42 | | | | | | | | | | | |
| | K | A | L | V | E | I | C | A | E | L | E | | | | | | | | | | | |
| aa 33-43 | 33 | 34 | 35 | 36 | 37 | 38 | 39 | 40 | 41 | 42 | 43 | | | | | | | | | | | |
| | A | L | V | E | I | C | A | E | L | E | K | | | | | | | | | | | |
| aa 34-44 | 34 | 35 | 36 | 37 | 38 | 39 | 40 | 41 | 42 | 43 | 44 | | | | | | | | | | | |
| | L | V | E | I | C | A | E | L | E | K | E | | | | | | | | | | | |
| aa 35-45 | 35 | 36 | 37 | 38 | 39 | 40 | 41 | 42 | 43 | 44 | 45 | | | | | | | | | | | |
| | V | E | I | C | A | E | L | E | K | E | G | | | | | | | | | | | |
| aa 36-46 | 36 | 37 | 38 | 39 | 40 | 41 | 42 | 43 | 44 | 45 | 46 | | | | | | | | | | | |
| | E | I | C | A | E | L | E | K | E | G | K | | | | | | | | | | | |
| aa 37-47 | 37 | 38 | 39 | 40 | 41 | 42 | 43 | 44 | 45 | 46 | 47 | | | | | | | | | | | |
| | I | C | A | E | L | E | K | E | G | K | I | | | | | | | | | | | |
| aa 38-48 | 38 | 39 | 40 | 41 | 42 | 43 | 44 | 45 | 46 | 47 | 48 | | | | | | | | | | | |
| | C | A | E | L | E | K | E | G | K | I | S | | | | | | | | | | | |
| aa 39-49 | 39 | 40 | 41 | 42 | 43 | 44 | 45 | 46 | 47 | 48 | 49 | | | | | | | | | | | |
| | A | E | L | E | K | E | G | K | I | S | K | | | | | | | | | | | |
| aa 40-50 | 40 | 41 | 42 | 43 | 44 | 45 | 46 | 47 | 48 | 49 | 50 | | | | | | | | | | | |
| | E | L | E | K | E | G | K | I | S | K | I | | | | | | | | | | | |
| * Sequences of 12aa: | | | | | | | | | | | | | | | | | | | | | | |
| aa 30-41 | 30 | 31 | 32 | 33 | 34 | 35 | 36 | 37 | 38 | 39 | 40 | 41 | | | | | | | | | | |
| | K | I | K | A | L | V | E | I | C | A | E | L | | | | | | | | | | |
| aa 31-42 | 31 | 32 | 33 | 34 | 35 | 36 | 37 | 38 | 39 | 40 | 41 | 42 | | | | | | | | | | |
| | I | K | A | L | V | E | I | C | A | E | L | E | | | | | | | | | | |
| aa 32-43 | 32 | 33 | 34 | 35 | 36 | 37 | 38 | 39 | 40 | 41 | 42 | 43 | | | | | | | | | | |
| | K | A | L | V | E | I | C | A | E | L | E | K | | | | | | | | | | |
| aa 33-44 | 33 | 34 | 35 | 36 | 37 | 38 | 39 | 40 | 41 | 42 | 43 | 44 | | | | | | | | | | |
| | A | L | V | E | I | C | A | E | L | E | K | E | | | | | | | | | | |
| aa 34-45 | 34 | 35 | 36 | 37 | 38 | 39 | 40 | 41 | 42 | 43 | 44 | 45 | | | | | | | | | | |
| | L | V | E | I | C | A | E | L | E | K | E | G | | | | | | | | | | |
| aa 35-46 | 35 | 36 | 37 | 33 | 39 | 40 | 41 | 42 | 43 | 44 | 45 | 46 | | | | | | | | | | |
| | V | E | I | C | A | E | L | E | K | E | G | K | | | | | | | | | | |
| aa 36-47 | 36 | 37 | 38 | 39 | 40 | 41 | 42 | 43 | 44 | 45 | 46 | 47 | | | | | | | | | | |
| | E | I | C | A | E | L | E | K | E | G | K | I | | | | | | | | | | |
| aa 37-48 | 37 | 38 | 39 | 40 | 41 | 42 | 43 | 44 | 45 | 46 | 47 | 48 | | | | | | | | | | |
| | I | C | A | E | L | E | K | E | G | K | I | S | | | | | | | | | | |
| aa 38-49 | 38 | 39 | 40 | 41 | 42 | 43 | 44 | 45 | 46 | 47 | 48 | 49 | | | | | | | | | | |
| | C | A | E | L | E | K | E | G | K | I | S | K | | | | | | | | | | |
| aa 39-50 | 39 | 40 | 41 | 42 | 43 | 44 | 45 | 46 | 47 | 48 | 49 | 50 | | | | | | | | | | |
| | A | E | L | E | K | E | G | K | I | S | K | I | | | | | | | | | | |
| * Sequences of 13aa: | | | | | | | | | | | | | | | | | | | | | | |
| aa 30-42 | 30 | 31 | 32 | 33 | 34 | 35 | 36 | 37 | 38 | 39 | 40 | 41 | 42 | | | | | | | | | |
| | K | I | K | A | L | V | E | I | C | A | E | L | E | | | | | | | | | |
| aa 31-43 | 31 | 32 | 33 | 34 | 35 | 36 | 37 | 38 | 39 | 40 | 41 | 42 | 43 | | | | | | | | | |
| | I | K | A | L | V | E | I | C | A | E | L | E | K | | | | | | | | | |
| aa 32-44 | 32 | 33 | 34 | 35 | 36 | 37 | 38 | 39 | 40 | 41 | 42 | 43 | 44 | | | | | | | | | |
| | K | A | L | V | E | I | C | A | E | L | E | K | E | | | | | | | | | |
| aa 33-45 | 33 | 34 | 35 | 36 | 37 | 38 | 39 | 40 | 41 | 42 | 43 | 44 | 45 | | | | | | | | | |
| | A | L | V | E | I | C | A | E | L | E | K | E | G | | | | | | | | | |
| aa 34-46 | 34 | 35 | 36 | 37 | 38 | 39 | 40 | 41 | 42 | 43 | 44 | 45 | 46 | | | | | | | | | |
| | L | V | E | I | C | A | E | L | E | K | E | G | K | | | | | | | | | |
| aa 35-47 | 35 | 36 | 37 | 38 | 39 | 40 | 41 | 42 | 43 | 44 | 45 | 46 | 47 | | | | | | | | | |
| | V | E | I | C | A | E | L | E | K | E | G | K | I | | | | | | | | | |
| aa 36-48 | 36 | 37 | 38 | 39 | 40 | 41 | 42 | 43 | 44 | 45 | 46 | 47 | 48 | | | | | | | | | |
| | E | I | C | A | E | L | E | K | E | G | K | I | S | | | | | | | | | |
| aa 37-49 | 37 | 38 | 39 | 40 | 41 | 42 | 43 | 44 | 45 | 46 | 47 | 48 | 49 | | | | | | | | | |
| | I | C | A | E | L | E | K | E | G | K | I | S | K | | | | | | | | | |
| aa 38-50 | 38 | 39 | 40 | 41 | 42 | 43 | 44 | 45 | 46 | 47 | 48 | 49 | 50 | | | | | | | | | |
| | C | A | E | L | E | K | E | G | K | I | S | K | I | | | | | | | | | |
| * Sequences of 14aa: | | | | | | | | | | | | | | | | | | | | | | |
| aa 30-43 | 30 | 31 | 32 | 33 | 34 | 35 | 36 | 37 | 38 | 39 | 40 | 41 | 42 | 43 | | | | | | | | |
| | K | I | K | A | L | V | E | I | C | A | E | L | E | K | | | | | | | | |
| aa 31-44 | 31 | 32 | 33 | 34 | 35 | 36 | 37 | 38 | 39 | 40 | 41 | 42 | 43 | 44 | | | | | | | | |
| | I | K | A | L | V | E | I | C | A | E | L | E | K | E | | | | | | | | |
| aa 32-45 | 32 | 33 | 34 | 35 | 36 | 37 | 38 | 39 | 40 | 41 | 42 | 43 | 44 | 45 | | | | | | | | |
| | K | A | L | V | E | I | C | A | E | L | E | K | E | G | | | | | | | | |
| aa 33-46 | 33 | 34 | 35 | 36 | 37 | 38 | 39 | 40 | 41 | 42 | 43 | 44 | 45 | 46 | | | | | | | | |
| | A | L | V | E | I | C | A | E | L | E | K | E | G | K | | | | | | | | |
| aa 34-47 | 34 | 35 | 36 | 37 | 38 | 39 | 40 | 41 | 42 | 43 | 44 | 45 | 46 | 47 | | | | | | | | |
| | L | V | E | I | C | A | E | L | E | K | E | G | K | I | | | | | | | | |
| aa 35-48 | 35 | 36 | 37 | 38 | 39 | 40 | 41 | 42 | 43 | 44 | 45 | 46 | 47 | 48 | | | | | | | | |
| | V | E | I | C | A | E | L | E | K | E | G | K | I | S | | | | | | | | |
| aa 36-49 | 36 | 37 | 38 | 39 | 40 | 41 | 42 | 43 | 44 | 45 | 46 | 47 | 48 | 49 | | | | | | | | |
| | E | I | C | A | E | L | E | K | E | G | K | I | S | K | | | | | | | | |
| aa 37-50 | 37 | 38 | 39 | 40 | 41 | 42 | 43 | 44 | 45 | 46 | 47 | 48 | 49 | 50 | | | | | | | | |
| | I | C | A | E | L | E | K | E | G | K | I | S | K | I | | | | | | | | |
| * Sequences of 15aa: | | | | | | | | | | | | | | | | | | | | | | |
| aa 30-44 | 30 | 31 | 32 | 33 | 34 | 35 | 36 | 37 | 38 | 39 | 40 | 41 | 42 | 43 | 44 | | | | | | | |
| | K | I | K | A | L | V | E | I | C | A | E | L | E | K | E | | | | | | | |
| aa 31-45 | 31 | 32 | 33 | 34 | 35 | 36 | 37 | 38 | 39 | 40 | 41 | 42 | 43 | 44 | 45 | | | | | | | |
| | I | K | A | L | V | E | I | C | A | E | L | E | K | E | G | | | | | | | |
| aa 32-46 | 32 | 33 | 34 | 35 | 36 | 37 | 38 | 39 | 40 | 41 | 42 | 43 | 44 | 45 | 46 | | | | | | | |
| | K | A | L | V | E | I | C | A | E | L | E | K | E | G | K | | | | | | | |
| aa 33-47 | 33 | 34 | 35 | 36 | 37 | 38 | 39 | 40 | 41 | 42 | 43 | 44 | 45 | 46 | 47 | | | | | | | |
| | A | L | V | E | I | C | A | E | L | E | K | E | G | K | I | | | | | | | |
| aa 34-48 | 34 | 35 | 36 | 37 | 38 | 39 | 40 | 41 | 42 | 43 | 44 | 45 | 46 | 47 | 48 | | | | | | | |
| | L | V | E | I | C | A | E | L | E | K | E | G | K | I | S | | | | | | | |
| aa 35-49 | 35 | 36 | 37 | 38 | 39 | 40 | 41 | 42 | 43 | 44 | 45 | 46 | 47 | 48 | 49 | | | | | | | |
| | V | E | I | C | A | E | L | E | K | E | G | K | I | S | K | | | | | | | |
| aa 36-50 | 36 | 37 | 38 | 39 | 40 | 41 | 42 | 43 | 44 | 45 | 46 | 47 | 48 | 49 | 50 | | | | | | | |
| | E | I | C | A | E | L | E | K | E | G | K | I | S | K | I | | | | | | | |
| * Sequences of 16aa: | | | | | | | | | | | | | | | | | | | | | | |
| aa 30-45 | 30 | 31 | 32 | 33 | 34 | 35 | 36 | 37 | 38 | 39 | 40 | 41 | 42 | 43 | 44 | 45 | | | | | | |
| | K | I | K | A | L | V | E | I | C | A | E | L | E | K | E | G | | | | | | |
| aa 31-46 | 31 | 32 | 33 | 34 | 35 | 36 | 37 | 38 | 39 | 40 | 41 | 42 | 43 | 44 | 45 | 46 | | | | | | |
| | I | K | A | L | V | E | I | C | A | E | L | E | K | E | G | K | | | | | | |
| aa 32-47 | 32 | 33 | 34 | 35 | 36 | 37 | 38 | 39 | 40 | 41 | 42 | 43 | 44 | 45 | 46 | 47 | | | | | | |
| | K | A | L | V | E | I | C | A | E | L | E | K | E | G | K | I | | | | | | |
| aa 33-48 | 33 | 34 | 35 | 36 | 37 | 38 | 39 | 40 | 41 | 42 | 43 | 44 | 45 | 46 | 47 | 48 | | | | | | |
| | A | L | V | E | I | C | A | E | L | E | K | E | G | K | I | S | | | | | | |
| aa 34-49 | 34 | 35 | 36 | 37 | 38 | 39 | 40 | 41 | 42 | 43 | 44 | 45 | 46 | 47 | 48 | 49 | | | | | | |
| | L | V | E | I | C | A | E | L | E | K | E | G | K | I | S | K | | | | | | |
| aa 35-50 | 35 | 36 | 37 | 38 | 39 | 40 | 41 | 42 | 43 | 44 | 45 | 46 | 47 | 48 | 49 | 50 | | | | | | |
| | V | E | I | C | A | E | L | E | K | E | G | K | I | S | K | I | | | | | | |
| * Sequences of 17aa: | | | | | | | | | | | | | | | | | | | | | | |
| aa 30-46 | 30 | 31 | 32 | 33 | 34 | 35 | 36 | 37 | 38 | 39 | 40 | 41 | 42 | 43 | 44 | 45 | 46 | | | | | |
| | K | I | K | A | L | V | E | I | C | A | E | L | E | K | E | G | K | | | | | |
| aa 31-47 | 31 | 32 | 33 | 34 | 35 | 36 | 37 | 38 | 39 | 40 | 41 | 42 | 43 | 44 | 45 | 46 | 47 | | | | | |
| | I | K | A | L | V | E | I | C | A | E | L | E | K | E | G | K | I | | | | | |
| aa 32-48 | 32 | 33 | 34 | 35 | 36 | 37 | 38 | 39 | 40 | 41 | 42 | 45 | 44 | 45 | 46 | 47 | 48 | | | | | |
| | K | A | L | V | E | I | C | A | E | L | E | K | E | G | K | I | S | | | | | |
| aa 33-49 | 33 | 34 | 35 | 36 | 37 | 38 | 39 | 40 | 41 | 42 | 43 | 44 | 45 | 46 | 47 | 48 | 49 | | | | | |
| | A | L | V | E | I | C | A | E | L | E | K | E | G | K | I | S | K | | | | | |
| aa 34-50 | 34 | 35 | 36 | 37 | 38 | 39 | 40 | 41 | 42 | 43 | 44 | 45 | 46 | 47 | 48 | 49 | 50 | | | | | |
| | L | V | E | I | C | A | E | L | E | K | E | G | K | I | S | K | I | | | | | |
| * Sequences of 18aa: | | | | | | | | | | | | | | | | | | | | | | |
| aa 30-47 | 30 | 31 | 32 | 33 | 34 | 35 | 36 | 37 | 38 | 39 | 40 | 41 | 42 | 43 | 44 | 45 | 46 | 47 | | | | |
| | K | I | K | A | L | V | E | I | C | A | E | L | E | K | E | G | K | I | | | | |
| aa 31-48 | 31 | 32 | 33 | 34 | 35 | 36 | 37 | 38 | 39 | 40 | 41 | 42 | 43 | 44 | 45 | 46 | 47 | 48 | | | | |
| | I | K | A | L | V | E | I | C | A | E | L | E | K | E | G | K | I | S | | | | |
| aa 32-49 | 32 | 33 | 34 | 35 | 36 | 37 | 38 | 39 | 40 | 41 | 42 | 43 | 44 | 45 | 46 | 47 | 48 | 49 | | | | |
| | K | A | L | V | E | I | C | A | E | L | E | K | E | G | K | I | S | K | | | | |
| aa 33-50 | 33 | 34 | 35 | 36 | 37 | 38 | 39 | 40 | 41 | 42 | 43 | 44 | 45 | 46 | 47 | 48 | 49 | 50 | | | | |
| | A | L | V | E | I | C | A | E | L | E | K | E | G | K | I | S | K | I | | | | |
| * Sequences of 19aa: | | | | | | | | | | | | | | | | | | | | | | |
| aa 30-48 | 30 | 31 | 32 | 33 | 34 | 35 | 36 | 37 | 38 | 39 | 40 | 41 | 42 | 43 | 44 | 45 | 46 | 47 | 48 | | | |
| | K | I | K | A | L | V | E | I | C | A | E | L | E | K | E | G | K | I | S | | | |
| aa 31-49 | 31 | 32 | 33 | 34 | 35 | 36 | 37 | 38 | 39 | 40 | 41 | 42 | 43 | 44 | 45 | 46 | 47 | 48 | 49 | | | |
| | I | K | A | L | V | E | I | C | A | E | L | E | K | E | G | K | I | S | K | | | |
| aa 32-50 | 32 | 33 | 34 | 35 | 36 | 37 | 38 | 39 | 40 | 41 | 42 | 43 | 44 | 45 | 46 | 47 | 48 | 49 | 50 | | | |
| | K | A | L | V | E | I | C | A | E | L | E | K | E | G | K | I | S | K | I | | | |
| * Sequences of 20aa: | | | | | | | | | | | | | | | | | | | | | | |
| aa 30-49 | 30 | 31 | 32 | 33 | 34 | 35 | 36 | 37 | 38 | 39 | 40 | 41 | 42 | 43 | 44 | 45 | 46 | 47 | 48 | 49 | | |
| | K | I | K | A | L | V | E | I | C | A | E | L | E | K | E | G | K | I | S | K | | |
| aa 31-50 | 31 | 32 | 33 | 34 | 35 | 36 | 37 | 38 | 39 | 40 | 41 | 42 | 43 | 44 | 45 | 46 | 47 | 48 | 49 | 50 | | |
| | I | K | A | L | V | E | I | C | A | E | L | E | K | E | G | K | I | S | K | I | | |
| * Sequences of 21aa: | | | | | | | | | | | | | | | | | | | | | | |
| aa 30-50 | 30 | 31 | 32 | 33 | 34 | 35 | 36 | 37 | 38 | 39 | 40 | 41 | 42 | 43 | 44 | 45 | 46 | 47 | 48 | 49 | 50 | |
| | K | I | K | A | L | V | E | I | C | A | E | L | E | K | E | G | K | I | S | K | I | |

### EXAMPLE NO. 6

### Detection of Resistance Mutations on The Proviral DNA Isolated from the Peripheral Blood Mononuclear Cells (PBMCs) of Patient 201#5

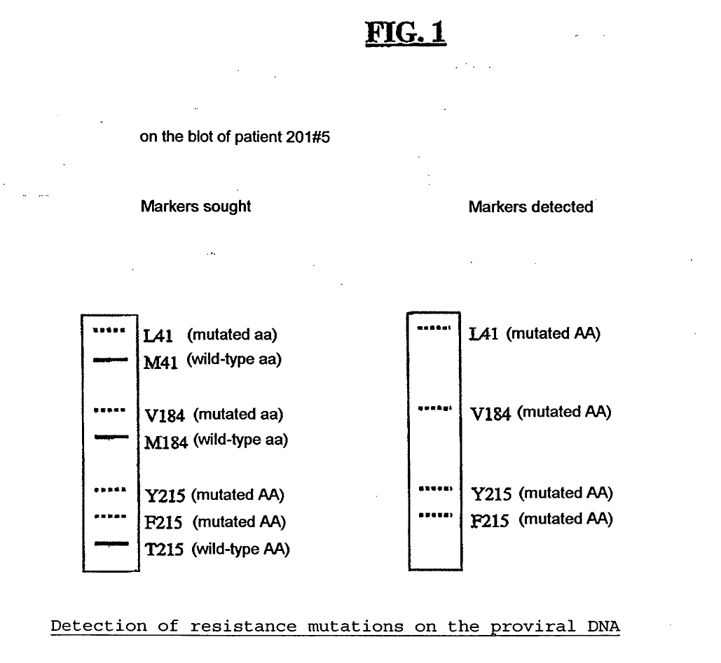

In patient 201#5 treated with NRTIs, detection of resistance mutations was carried out in the proviral DNA using an LiPA HIV-1-RT assay (see example 3). The LiPA assay allows the definition of point-mutated amino acids within a given sequence and not that of nucleic acid sequences. The method for detecting the resistance mutations on the DNA is summarized in FIG. 1.

Using this assay, we detected, in this patient, the mutations 41, 184 and 215.

The RT sequence of the sample from patient 201#5 is thus:
Xx41(L)Xx184(V)Xx215(Y or F)Xx
in which Xx represents a reference amino acid sequence.

### EXAMPLE NO. 7

### Ex Vivo Evaluation of the Immunogenicity of Mutation 41 (M>L) on the PBMCs from Patient 201#5

The recognition of RT mutation 41 (M>L) by gamma-IFN-producing CD8 cells is studied in patient 201#5 treated with NRTIs. Mutation 41 had been detected on the proviral DNA isolated from these same PBMCs (see example 6). The recognition of the wild-type and mutated peptides 33-41 was evaluated by ELISpot.

| | Wild-type | Mutated |
|---|---|---|
| Peptide 33-41 | ALVEICTEM | ALVEICTEL |
| SFC/10⁶ PBMCs | 3 | 95 |

The frequency of recognition of the mutated peptide 33-41 is 95 SFC/10⁶ PBMCs and 3 SFC/10⁶ PBMCs for the wild-type peptide 33-41.

### EXAMPLE NO. 8

### Structure of a Lipopeptide

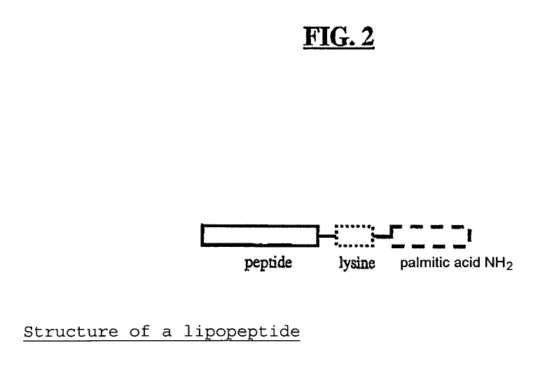

The general structure of a lipopeptide is described in FIG. 2.

A lipopeptide is made up of a palmitic acid of formula C₁₆H₃₂O₂ or described in the text as C₁₅H₃₁—COOH, of a lysine residue (basic amino acid added to the N-terminal end of the peptide) and of a peptide, the structure of which is chosen from the described sequences.

For peptide 30-50 (sequence B):

| | | | | | | | | | | | | | | | |
|---|---|---|---|---|---|---|---|---|---|---|---|---|---|---|---|
| aa | 30 | 31 | 32 | 33 | 34 | 35 | 36 | 37 | 38 | 39 | 40 | 41 | 42 | 43 | 44 |
| | K | I | K | A | L | V | E | I | C | M2 | E | M3 | E | K | E |
| | 45 | 46 | 47 | 48 | 49 | 50 | | | | | | | | | |
| | G | K | I | S | K | I | | | | | | | | | |

in which M2 (aa39) may be X39 or may be mutated to A, K, N, P, M or S; M3 (aa41) may be X41 or may be mutated to L; at least one of M2 and M3 representing an amino acid carrying a mutation.

Example of lipopeptide constructed with sequence 30-50 including resistance mutations 39 and 41 induced by NRTIs

| | | | | | | | | | | | | | | | |
|---|---|---|---|---|---|---|---|---|---|---|---|---|---|---|---|
| aa | 30 | 31 | 32 | 33 | 34 | 35 | 36 | 37 | 38 | 39 | 40 | 41 | 42 | 43 | 44 |
| | K | I | K | A | L | V | E | I | C | A | E | L | E | K | E |
| | 45 | 46 | 47 | 48 | 49 | 50 | | | | | | | | | |
| | G | K | I | S | K | I | | | | | | | | | |

This peptide is referred to as aa30-aa50 (aa50=I).

In this example, the lysine is added to amino acid 50, which is an isoleucine (I). The amine function of the lysine side chain is modified with a palmitic acid and the C-terminal amino acid is amidated according to the following reactions:

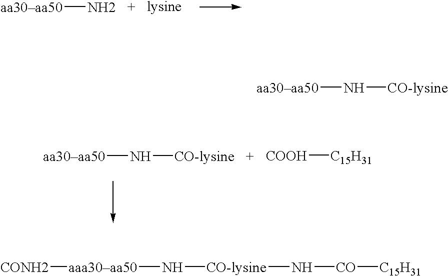

This structure varies according to the industrial processes. Thus, the lysine may be added to the C-terminal end of the peptide.

This structure is then purified and conditioned according to industrial processes corresponding to the conditions required for obtaining clinical batches before use for immunization in humans.

1-32. (canceled)

33. A method of treating an infectious and/or tumor pathology comprising:
treating a subject with a resistance mutation-inducing anti-infectious and/or antitumor chemotherapy agent; and
treating the subject with a vaccine directed against the resistance mutation;
wherein the treatment with the resistance-mutation inducing agent and the treatment with the vaccination occur simultaneously, separately or sequentially over time.

34. An agent comprising at least:
a first component comprising a resistance mutation-inducing anti-infectious and/or antitumor chemotherapy agent; and
a second component comprising a vaccine directed against the resistance mutation.

35. The agent of claim 34, further defined as comprising:
at least one medicinal product of the NRTI and/or NNRTI family; and
at least one peptide of 8 to 80 amino acids of the HIV reverse transcriptase sequence, comprising at least one mutation by simple or multiple substitution and/or insertion with respect to the established reference sequences of reverse transcriptase, in the absence of any treatment, which mutation is observed subsequent to treatment with NRTIs and/or NNRTIs in patients infected with HIV, the peptides being capable of inducing a response mediated by T lymphocytes specific for this mutated sequence of HIV reverse transcriptase with the exception of the peptides of sequence Z₁-Y-V-D-D-Z₂, Z₁-Y-I-D-D-Z₂ and Z₁-Y-L-D-D-Z₂, with Z₁ and Z₂ being at least any one amino acid.

36. A peptide of 8 to 80 amino acids of the HIV reverse transcriptase sequence, comprising at least one mutation by simple or multiple substitution and/or insertion with respect to the established reference sequences of reverse transcriptase, in the absence of any treatment, which mutation is observed subsequent to treatment with NRTIs and/or NNRTIs in patients infected with HIV, the peptides being capable of inducing a response mediated by T lymphocytes specific for this mutated sequence of HIV reverse transcriptase with the exception of the peptides of sequence Z₁-Y-V-D-D-Z₂, Z₁-Y-1-D-D-Z₂ and Z₁-Y-L-D-D-Z₂, with Z₁ and Z₂ being at least any one amino acid.

37. The peptide of claim 36, further defined as comprising from 15 to 50 amino acids.

38. The peptide of claim 36, further comprising one or more neutral amino acids that solubilize or stabilize the peptide.

39. The peptide of claim 36, further defined as dispersed in a pharmaceutically acceptable excipient.

40. The peptide of claim 36, further defined as adapted for use as a vaccine.

41. A vaccine composition comprising:
at least one peptide of 8 to 80 amino acids of the HIV reverse transcriptase sequence, comprising at least one mutation by simple or multiple substitution and/or insertion with respect to the established reference sequences of reverse transcriptase, in the absence of any treatment, which mutation is observed subsequent to treatment with NRTIs and/or NNRTIs in patients infected with HIV, the peptides being capable of inducing a response mediated by T lymphocytes specific for this mutated sequence of HIV reverse transcriptase with the exception of the peptides of sequence Z₁-Y-V-D-D-Z₂, Z₁-Y-1-D-D-Z₂ and Z₁-Y-L-D-D-Z₂, with Z₁ and Z₂ being at least any one amino acid; or
at least one nucleic acid encoding such a peptide.

42. The vaccine composition of claim 41, further defined as comprising an immunomodulator.

43. The vaccine composition of claim 41, further defined as comprising at least one adjuvant.

44. The vaccine composition of claim 41, further defined as comprising at least one peptide of 8 to 80 amino acids of the HIV reverse transcriptase sequence, comprising at least one mutation by simple or multiple substitution and/or insertion with respect to the established reference sequences of reverse transcriptase, in the absence of any treatment, which mutation is observed subsequent to treatment with NRTIs and/or NNRTIs in patients infected with HIV, the peptides being capable of inducing a response mediated by T lymphocytes specific for this mutated sequence of HIV reverse transcriptase with the exception of the peptides of sequence Z₁-Y-V-D-D-Z₂, Z₁-Y-1-D-D-Z₂ and Z₁-Y-L-D-D-Z₂, with Z₁ and Z₂ being at least any one amino acid.

45. The vaccine composition of claim 44, wherein the at least one peptide is coupled to at least one lipopeptide.

46. The vaccine composition of claim 44, wherein the at least on peptide is coupled to at least one lipoprotein.

47. The vaccine composition of claim 44, wherein the at least one peptide is coupled to a microparticle or nanoparticle.

48. The vaccine composition of claim 44, wherein the at least one peptide is coupled to at least one liposome.

49. The vaccine composition of claim 44, wherein the at least one peptide is coupled to at least one niosome.

50. The vaccine composition of claim 41, further defined as comprising at least one nucleic acid encoding at least one peptide of 8 to 80 amino acids of the HIV reverse transcriptase sequence, comprising at least one mutation by simple or multiple substitution and/or insertion with respect to the established reference sequences of reverse transcriptase, in the absence of any treatment, which mutation is observed subsequent to treatment with NRTIs and/or NNRTIs in patients infected with HIV, the peptides being capable of inducing a response mediated by T lymphocytes specific for this mutated sequence of HIV reverse transcriptase with the exception of the peptides of sequence Z₁-Y-V-D-D-Z₂, Z₁-Y-I-D-D-Z₂ and Z₁-Y-L-D-D-Z₂, with Z₁ and Z₂ being at least any one amino acid.

51. The vaccine composition of claim 50, wherein the at least one nucleic acid is further defined as comprised in at least one recombinant virus.

52. The vaccine composition of claim 50, wherein the at least one nucleic acid is further defined as comprised in at least one attenuated or inactivated virus.

53. The vaccine composition of claim 50, wherein the at least one nucleic acid is further defined as comprised in at least one recombinant viral vector.

54. The vaccine composition of claim 50, wherein the at least one nucleic acid is further defined as comprised in at least one recombinant bacterium.

55. The vaccine composition of claim 50, wherein the at least one nucleic acid is further defined as comprised in at least one recombinant bacterial vector.

56. The vaccine composition of claim 50, wherein the at least one nucleic acid is further defined as comprised in at least one antigen-presenting cell.

57. The vaccine composition of claim 50, wherein the at least one nucleic acid is further defined as at least one naked DNA.

58. A method for preventing or treating an HIV virus infection comprising inducing a specific T immune response in response to the administration of a vaccine composition comprising:
at least one peptide of 8 to 80 amino acids of the HIV reverse transcriptase sequence, comprising at least one mutation by simple or multiple substitution and/or insertion with respect to the established reference sequences of reverse transcriptase, in the absence of any treatment, which mutation is observed subsequent to treatment with NRTIs and/or NNRTIs in patients infected with HIV, the peptides being capable of inducing a response mediated by T lymphocytes specific for this mutated sequence of HIV reverse transcriptase with the exception of the peptides of sequence Z₁-Y-V-D-D-Z₂, Z₁-Y-I-D-D-Z₂ and Z₁-Y-L-D-D-Z₂, with Z₁ and Z₂ being at least any one amino acid; or
at least one nucleic acid encoding such a peptide.

59. The method of claim 58, characterized in that the mutation in the HIV virus is located in the reverse transcriptase enzyme.

60. A peptide sequence of 8 to 20 amino acids, further defined as a T cell epitope sequence specific for a vaccine peptide sequence.

61. The peptide sequence of claim 60, wherein the peptide sequence is a peptide sequence of 8 to 20 contiguous amino acids chosen from the peptide sequences A to K having at least one mutation Mp.

62. The peptide sequence of claim 61, wherein the peptide sequence is a peptide sequence of 8 to 10 contiguous amino acids chosen from the peptide sequences A to K having at least one mutation Mp.

63. The peptide sequence of claim 62, wherein the peptide sequence is a peptide sequence of 9 contiguous amino acids chosen from the peptide sequences A to K having at least one mutation Mp.

64. The peptide sequence of claim 60, further defined as comprised in a diagnostic composition.

65. A method comprising obtaining a peptide of 8 to 20 amino acids, further defined as a T cell specific epitope sequence, and determining and/or quantifying T cells specific for the epitope sequence.
